(19) United States
(12) Patent Application Publication
(10) Pub. No.: US 2015/0110847 A1
(43) Pub. Date: Apr. 23, 2015

(54) Substrate Mediated Enzymes Prodrug Therapy

(71) Applicant: Aarhus Universitet, Aarhus C (DK)

(72) Inventors: Alexander Zelikin, Aarhus N (DK); Betina Fejerskov Kristensen, Tranbjerg J (DK)

(73) Assignee: Aarhus Universitet, Aarhus C (DK)

(21) Appl. No.: 14/401,980

(22) PCT Filed: May 21, 2013
(86) PCT No.: PCT/DK2013/050152

§ 371 Date: Nov. 18, 2014

(30) Foreign Application Priority Data
May 18, 2012 (DK) PA 2012 70265

(51) Int. Cl. A61L 31/14; A61L 31/04; A61L 27/16; A61L 27/52; A61L 27/34; A61K 31/7048; A61L 31/10

(52) U.S. Cl. CPC A61L 31/145; A61K 31/7048; A61L 31/048; A61L 31/10; A61L 27/52; A61L 27/34; A61L 27/16; A61L 2420/08; A61L 2420/06; A61L 2300/608; A61L 2300/42; A61L 2300/254
USPC 424/423; 514/27; 424/94.61; 435/375

(57) ABSTRACT

A method is provided for administering an active drug agent to cells adhering and/or adjacent to a substrate, said substrate comprising one or more immobilized enzymes, which are capable of converting an inert prodrug into an active drug agent, said method comprising providing at least one inactive prodrug to said substrate. Also a kit is provided which comprise a substrate comprising one or more immobilized enzymes capable of converting an inert prodrug into an active drug agent, and at least one inert prodrug. A stent is also provided, which comprise a substrate comprising one or more immobilized enzymes capable of converting an inert prodrug into an active drug agent.

### FIELD OF INVENTION

The present invention relates to substrate mediated enzymes prodrug therapy (SMEPT), which allows the delivery of an active drug agent to a specific target site, by administrating an inert prodrug, which is converted to active drug by an enzyme in situ within a substrate, such that cells adhering to the substrate and neighbouring cells are exposed to the drug, without exposing more distantly located cells to the action of the drugs.

### BACKGROUND OF INVENTION

Administration of an active agent to a target tissue, such as the delivery of a therapeutic drug to a specific bodily tissue, often necessitates the administration of large amounts of drug to permit a therapeutically sufficient local concentration at the target site, in order to obtain a high absolute accretion of the therapeutic agent at the target site to permit long duration of uptake, binding, or exposure. High dosages result in high background levels, however, which may result in undesired harm to healthy cells and tissue. One attempt to overcome problems of non-specific delivery of cytotoxic therapeutic agents has involved the use of inert prodrugs, which are converted to therapeutically active agents at the target site. In antibody-directed enzyme-prodrug therapy (ADEPT), an enzyme capable of converting an inert prodrug into an active drug agent, is coupled to a tumour-specific antibody. The antibody linked enzyme is injected to the blood, resulting in selective binding of the enzyme in the tumour. A prodrug is then administrated into the blood circulation and is converted to an active cytotoxic drug by the enzyme, only within the tumour, where the enzyme is present. Selectivity is achieved by the tumour specificity of the antibody and by delaying prodrug administration until there is a large differential between tumour and normal tissue enzyme levels. The ADEPT method requires the availability of highly specific antibodies, which are not always available. Thus, for each such therapy, specific antibodies must be developed, and for each antibody, the immunoreactivity can be affected by the enzyme-linkage. Moreover, the epitopes presented in the tumour tissue may change during tumour progression, which would again necessitate the use of several different antibody-enzyme couples during the course of treatment.

### SUMMARY OF INVENTION

A main objective of the present invention is to provide a substrate mediated enzymes prodrug therapy (SMEPT), which allows the delivery of an active drug agent to a specific target site by administrating an inert prodrug, which is converted to an active drug agent in situ within a substrate by an enzyme confined to that specific substrate, such that cells adhering to the substrate and neighbouring cells are exposed to the drug, without exposing more distantly located cells to the action of the drugs. In particular, the enzymes of the present invention are not generally linked to an antibody or a fragment thereof.

In one aspect, the present invention relates to a method of administering an active drug agent to cells adhering to or adjacent to a substrate, said substrate comprising one or more immobilized enzymes, which are capable of converting an inert prodrug into an active drug agent, said method comprising providing at least one inactive prodrug to said substrate.

In another aspect, the invention relates to a method of administering an active drug agent to cells adhering and/or adjacent to a substrate by in situ production of the drug within said substrate, wherein said substrate comprises one or more immobilized enzymes, which are capable of converting an inert prodrug into an active drug agent.

In a third aspect, the present invention pertains to a method of treating a disease in an individual, said method comprising localizing a substrate comprising one or more immobilized enzymes capable of converting an inert prodrug into an active drug agent to a target site for treatment, and systemically administering an efficient amount of said inert prodrug to said individual, thereby bringing said prodrug into contact with said one or more enzymes and thereby converting said inert prodrug into an active drug at the target site for treatment.

In a fourth aspect, the invention relates to a method of treating atherosclerosis, said method comprising inserting into a blood vessel a vascular stent or a vascular graft, comprising a substrate comprising one or more immobilized enzymes capable of converting an inert prodrug into an active drug agent, and administering an inert prodrug, which is converted into an active anti-coagulating drug agent.

In certain embodiment of the aspects mentioned above, the substrate is a hydrogel, for example a polyvinyl alcohol (PVA) hydrogel. However, the substrate may be any type of polymer, polymer thin film or porous solid matrice. The substrate may also be selected from but not limited to poly-N-isopropylacrylamide (pNIPAAM), Polyethylene glycol (PEG), and Alginate.

In another embodiment, the substrate is a material having a surface comprising sequential polymer depositions (also known as a layer-by-layer coating). This layer-by-layer coating is for example alternating layers of the polymer pairs poly(L-lysine) (PLL)/Alginate, PLL/poly(L-glutamic acid) (PGA), thiol-modified poly(methacrylic acid) (PMAsh)/poly(N-vinylpyrrolidone) (PVP) or poly(allylamine hydrochloride) (PAH)/poly(acrylic acid) (PAA).

The substrate of the above-mentioned aspects could constitute or be part of a stent, a vascular graft, or any implantable device, or a scaffold for tissue engineering.

The immobilized enzyme is in one embodiment a glucuronidase, a glycosidase or a beta-lactamase; for example β-glucuronidase

The prodrug is in one embodiment a glucuronide-conjugated prodrug. The active drug agent is for example a growth hormone, a cytotoxic drug agent, an anti-inflammatory drug agent, an antiviral drug agent, or an anti-platelet aggregation drug agent, such as clopidogrel. Thus, in one embodiment, the enzyme is β-glucuronidase and said prodrug is a glucuronide prodrug.

The methods as defined in the aspects and embodiments above are not confined to single prodrugs, but may include administering multiple prodrugs, such as 1, 2, 3, 4, 5 or more different inert prodrugs. The same methods are also not confined to single enzymes but may include substrates comprising 1, 2, 3, 4, 5 or more different enzymes.

The prodrug is converted to active drug agent by one or more enzymes in the target substrate, and thus in the methods of the invention, the prodrug can be provided temporally only when the action of the active drug agent is required to be administered to cells adhering and/or adjacent to the substrate. Also, the amount of active drug produced at the site of the substrate reflects to amount of prodrug administered, and thus in the methods of the invention, the amount of provided inert prodrug reflects the level of active drug agent to be administered to cells adhering and/or adjacent to the substrate.

In a further aspect, the present invention relates to a kit comprising a substrate comprising one or more immobilized enzymes capable of converting an inert prodrug into an active drug agent, and said inert prodrug.

The invention also in one aspect relates to a substrate comprising one or more immobilized enzymes capable of converting an inert prodrug into an active drug agent for use in medicine. For example, the invention in one aspect relates to a substrate comprising one or more immobilized enzymes capable of converting an inert prodrug into an active drug agent for use in the treatment of cardiovascular disorders.

In another aspect, the invention provides an implantable device comprising a substrate comprising one or more immobilized enzymes capable of converting an inert prodrug into an active drug agent. The implantable device is for example a stent comprising a substrate comprising one or more immobilized enzymes capable of converting an inert prodrug into an active drug agent. Thus, in one aspect, the invention relates to a stent or a vascular graft, comprising a substrate comprising one or more immobilized enzymes capable of converting an inert prodrug into an active drug agent. Such stent is for example a cardiovascular stent, a bowel stent, a ureteral stent, a prostatic stent, an esophageal stent, a biliary stent, a stent graft, or a vascular graft.

Moreover, the invention relates to an implantable device for use in medicine, wherein said implantable device comprises a substrate comprising one or more immobilized enzymes capable of converting an inert prodrug into an active drug agent.

### DESCRIPTION OF DRAWINGS

FIG. 1: Schematic illustration of the concept of Surface Mediated Enzyme prodrug Therapy (SMEPT). A cell culture substrate contains an enzyme for conversion of an inactive prodrug into an active product which is then internalized by the adhering cells or adjacent tissues. By design, in situ generation of the active product is externally initiated at the moment of choice with several independent arms of control over the rate and the overall amount of the produced product available “postimplantation”.

FIG. 2. (A) Differential interference contrast (top) and fluorescence (bottom) microscopy images of the surface adhered μS PVA hydrogel thin films. The samples were prepared using PDMS molds with cubic 2 μm sized cavities, stabilized using 0.5 M sodium sulfate, washed with PBS and visualized in hydrated state. (B) Surface mediated enzyme prodrug therapy allows initiating generation of the product “postimplantation” at a desired time point, as evidenced by fluorescence evolution profiles obtained using β-Glu containing μS PVA hydrogel thin films and addition of the fluorogenic substrate at times t=0 and t=24 h. (C) Conversion of the fluorogenic substrate is achieved by the gel-immobilized enzyme, as verified by the levels of enzymatic activity measured in the coagulation salt, subsequent PBS wash and cell culture media wash, as well as registered using μS PVA hydrogel thin films. Experimental conditions (B): 1 g/L β-Glu in the polymer solution used for the production of hydrogels; [FG]: 5 μg/mL; (C) As above, 30 min reaction time.

FIG. 3. Design of SMEPT provides several arms of control over the rate of generation and the overall amount of the product generated by the enzyme containing substrates, all available “post-implantation”: concentration of the enzyme in the gel (A), concentration of the added substrate (B) and time of enzymatic conversion (C). Together, the methods allow controlling the amount of generated substrate over at least 3 orders of magnitude. Experimental conditions: (A) FG, 5 pg/mL, 30 min reaction time; (B): 1 g/L β-Glu in polymer solution, 30 min reaction time; (C) 1 g/L enzyme in polymer solution, initial concentration of FG: 1 (top), 0.1 (middle) and 0.01 (bottom) μg/mL, respectively.

FIG. 4. Utility of SMEPT in generation and cellular internalization of in situ produced product is verified through quantification of fluorescence of hepatic cells cultured on the μS PVA hydrogels. (A) Solution based generation and internalization of a fluorescent product (blue trace) and SMEPT (red trace) afforded comparable levels of fluorescence of cultured cells, as quantified using flow cytometry, while administration of the pro-drug in absence of the enzyme led to negligible change in the fluorescence of cultured cells (black trace). (B) Using concentration of the prodrug and time as tools of control over substrate mediated drug delivery, SMEPT reveals time- and dose-dependent internalization of the model fluorescent product generated in situ within the substrate for cell adhesion. Experimental conditions: (A) 1 g/L enzyme in the polymer solution; initial concentration of fluorescein diglucuronide, 2.5 μg/mL; incubation time 24 h; (B) 1 g/L enzyme in the polymer solution; initial concentration of fluorescein diglucuronide 0.25 (top) and 0.025 (bottom) 5 μg/mL.

FIG. 5: PVA hydrogels were assembled via uTM and using sodium sulfate coagulation baths with varied concentration of the sulfate. Hydrogels were stabilized by “salting out”-method in 1 h and 24 h using different Na2SO4-concentrations, thereafter put in PBS for 1 h and 24 h and visualized in hydrated state in PBS (40× magnification).

FIG. 6: The distribution of polymer within stabilization salt, PBS and the micro-structures (N=3)

FIG. 7. The retention of polymer within stabilized structures using different stabilization-time and -concentration (N=3).

FIG. 8. Activity of the b-Glu immobilized within μS PVA hydrogels as a function of concentration of sodium sulfate in a coagulation bath used for the preparation of the hydrogel.

FIG. 9: Topography analysis for μS PVA hydrogels prepared using coagulation baths with varied concentration of sodium sulfate.

FIG. 10: Young's moduli of μS PVA hydrogels prepared using coagulation baths with varied concentration of sodium sulfate.

FIG. 11. This figure introduces a method to quantify enzymatic activity during the assembly of μS PVA hydrogels as matrices for SMEPT. Enzymatic activity is quantified in the coagulation bath, subsequent PBS wash and finally within the μS PVA hydrogels.

FIG. 12: A,B. Enzymatic activity registered using μS PVA hydrogels prepared using solutions of 10 (left) and 35 (right) KDa PVA taken at varied concentration.

FIG. 13. Comparison of enzyme activity within hydrogel cubes of 4.5, 10 and 35 kDa taken at the same concentration.

FIG. 14: simply to show that we can make PVA hydrogels cell adhesive. Pristine hydrogels do not support cell adhesion and cells “stop” in their proliferation at the border of the gel. In contrast, RGD-functionalized gels are well suited as substrates for cell adhesion.

FIG. 15: Enzyme activity in μS PVA hydrogels prepared using polymers with differed molecular weight. Substrate conversion was initiated in samples incubated in PBS for 1 h (top, left), 24 h (top, right) and 7 days (bottom). Importance of this, further to the basic system characterization, lies in that we can make the SMEPT matrices with time-programmed duration, i.e. life-time.

FIG. 16: Enzyme activity for μS PVA hydrogels prepared using 35 KDa polymer sample with substrate conversion initiated at specified time points: 1 h, 24 h, 3 days and 7 days

FIG. 17: Viability of cells on μS hydrogels with enzyme and/or prodrug.

FIG. 18: A. Schematic illustration of Layer-by-Layer. B. For the four pH values of the enzyme solution, QCM-D frequency changes for the assembly of (PLUPGA)2.5 multilayers, the adsorption of the enzyme b-glu followed by the deposition of (PGA/PLL)2.

FIG. 19: Total change in frequency (A) and total change in dissipation (B) upon (PLL/PGA)2.5/b-glu/(PGA/PLL)2 film deposition using different pH for the b-glu adsorption. C) Change in frequency upon adsorption of b-glu to (PLUPGA)2.5 pre-coated crystals using different pH for the enzyme deposition. D) Enzyme activity in the form of converted prodrug (fluorescein di-glucuronide into fluorescein) using the biocatalytic films with the enzyme assembled at different pH. The negative control is fluorescence measured for (PLL PGA)4.5 films without enzyme. For all panels, the number of experiments n≧3. B.

FIG. 20: Enzyme activity in the form of converted prodrug along with frequency changes. All films are made with PLL as polycation. The polyanion used is written on the x-axis and the buffer used to introduce the enzyme is shown in parentheses. For each column, number of experiments N≧3.

FIG. 21: The cells were seeded on: β-glucuronidase containing LbL films added SN-38 glucuronide in solution at 1 μM giving SMEPT conditions (A), LbL films added β-glucuronidase and SN-38 glucuronide at 1 μM, both in solution mimicking EPT (B), LbL films added SN-38 at 1 μM (C), LbL films added SN-38 glucuronide at 1 μM (D), and R-Glucuronidase containing LbL films (E) (N=3).

FIG. 22: Controlled prodrug conversion with flow rate 0.03 dyn/cm2. (1 mL/h). Black datapoints represent chamber A, where FdG was led through to start with (as indicated on the x-axis), while red datapoints represent chamber B, where buffer was led through in the beginning (N=3).

FIG. 23: A) Prodrug conversion in flow at different rates of 0.15, 0.074, 0.03 dyn/cm2 (5, 2.5 and 1 mL/h) (N=3). B) Prodrug conversion in flow with different prodrug concentrations (N 3 for 50 μg/mL) or number of layers of incorporated enzyme incorporated (N =3).

FIG. 24: Cell viability after 4 h flow in chambers with (pro)drug of 1 μM. SN-38 glucuronide was added to chambers with 1× enzyme incorporated in the layers. SN-38 was added to chambers with no enzyme (N=3).

FIG. 25: Cell viability after 4 h flow in (connected) chambers with 1 μM SN-38 glucuronide (N=3).

FIG. 26: SMEPT using β-Galactosidase in a PVA hydrogel. Fluorogenic prodrug (resorufin galactopyranose, RGp) is used to illustrate flexibility of the SMEPT technology. The graph clearly shows that concentration of the enzyme within the hydrogel and concentration of the administered prodrug provide two independent tools to control the amount of the active drug product generated in unit time.

FIG. 27: SMEPT using β-Galactosidase in a PVA hydrogel. The graph shows that with appropriate choice of enzyme and prodrug concentrations, it is possible to achieve a constant rate of prodrug conversion (evidenced by a linear curve of product accumulation).

FIG. 28: SMEPT using β-Galactosidase in a PVA hydrogel. Enzymes immobilized in a substrate of the invention are stable over a prolonged period of time, and therefore allow storage. The data in this experiment show that PVA biocatalytic hydrogels can be stored at 4° C. (fridge) for at least 7 days with no decrease in activity of SMEPT. The shelf life is imperative for practical utility of the SMEPT system.

FIG. 29: SMEPT using β-Galactosidase in a PVA hydrogel. Similar to the experiments described above in FIGS. 25, characterization of biocatalytic hydrogels was performed under flow conditions. Flow rate and concentration of the prodrug provide two independent tools to control the amount of product generated in unit time. A high flow rate can be balanced by increasing the concentration of prodrug provided, whereas a low flow rate can be compensated by decreasing concentration of prodrug. Note, however, that prodrug concentration appears to be more powerful in defining prodrug conversion in unit time: 5-fold change in the flow rate provides a 2-fold change in the product concentration. At the same time, 10× change in prodrug concentration affords a corresponding 10× change in the amount of the generated product. Importantly, at each specific concentration and flow rate, the concentration of the active drug product remains constant. This is an important feature of surface mediated drug delivery.

FIG. 30: PVA hydrogels with immobilized β-glucuronidase are used to illustrate the possibility afforded by the SMEPT technology to synthesize at least 2 drugs with an independent control over concentration of each of them. In this experiment, 2 fluorogenic prodrugs were used, and as shown,it is possible to fill in the substrate matrix and synthesize one, the other, or indeed two drugs together in any desired concentration.

FIG. 31: Enzymatic conversion of different concentrations of the prodrug b-gal-NONOate by the enzyme g-galactosidase (N=3).

### DETAILED DESCRIPTION OF THE INVENTION

The present invention relates to substrate mediated enzymes prodrug therapy (SMEPT), which provides a simple, reliable and adjustable methodology of delivering active drugs to a specific target site, where an inert prodrug is converted to active drug at the target site, thereby avoiding any adverse side effects of the active drug in other locations. Generally, the present invention relates to a method, wherein one or more active drug agents are produced in situ within a substrate, such that cells adhering to the substrate and neighbouring cells are exposed to the drug, without exposing more distantly located cells to the action of the drugs. This allows the treatment of a specific subpopulation of cells, which adhere or are adjacent to the substrate, without affecting other non-adhering or non-adjacent cells.

The term “adjacent” as used herein for cells adjacent to a substrate, refers to cells, which are in the immediate vicinity of the substrate without being in direct contact with the substrate. However, due to for example diffusion of the converted active drug agent over small distances or translocation of the active drug from adhering cells to neighbouring cells, cells which are in the immediate vicinity of the substrate may also be exposed to the active drug.

In situ drug production is achieved by incorporating into the substrate one or more enzymes, which are capable of converting an inert prodrug to an active drug agent. Thus, when an inert prodrug is provided to the substrates, the embedded enzymes catalyse the conversion of the prodrug to an active drug agent within or at the substrate, whereby cells adhering and/or adjacent to the substrate are exposed to actions of the active drug agent. So, the present invention provides a method of administering an active drug agent to cells adhering and/or adjacent to a substrate by in situ production of the drug within or at the substrate, wherein the substrate comprises one or more immobilized enzymes, which are capable of converting an inert prodrug into an active drug agent.

Thus, in one aspect, the present invention provides a method of administering an active drug agent to cells adhering and/or adjacent to a substrate, said substrate comprising one or more immobilized enzymes, which are capable of converting an inert prodrug into an active drug agent, said method comprising the step of providing at least one inactive prodrug to said substrate. In another aspect, the invention provides a method of administering an active drug agent to cells adhering and/or adjacent to a substrate by in situ production of the drug within said substrate, wherein said substrate comprises one or more immobilized enzymes, which are capable of converting an inert prodrug into an active drug agent.

The methods can be used in order to administer an active drug agent to a subset of cells, without affecting non-adhering and non-adjacent cells. The method thus allows the administration of an active drug agent to a subpopulation of cells within a population of cells. In one embodiment, the method allows the administration of an active drug agent to specific cells, such as specific cells within a plurality of cells or within a multicellular organism or tissues of an organism without affecting non-targeted cells of the plurality of cells or organism or without affecting other tissues of the organism, respectively. For example, for treatment of a specific tissue within an organism, such as an animal or a human being, an implantable device consisting of or comprising a substrate of the present invention, can be inserted into that tissue, and the inert prodrug can be provided by systemic administration to the organism. Since the prodrug is inert, the cells of the organism will not be generally exposed to the active drug. However, cells or tissues adhering and/or adjacent to the substrate, which comprise enzymes capable of converting the prodrug to active drug, will be exposed to the active drug. In a similar fashion, within a plurality of cells, specific cells attached to a specific region of a surface substrate, said specific region comprising one or more immobilized enzymes, can be targeted without affected the remaining cells in the plurality of cells, which do not adhere (and are not adjacent to) to the specific region of the substrate surface where one or more enzymes are immobilized. This would allow in vitro culturing of cells, for example in petri dishes, flasks or other container or compartment, wherein a subset of the cells are administered a specific active drug agent, while the remaining cells are not administered such agent. The active drug agent could in this connection be a growth hormone or other growth factor or agent, which directs differentiation of the cells in the culture.

Thus, in one aspect, the present invention provides a method of treating a specific tissue or specific cells in a subject, such as an animal or human being, said method comprising localizing a substrate comprising one or more immobilized enzymes capable of converting an inert prodrug into an active drug agent within or adjacent to that specific tissue or specific cells, and systemically administering an efficient amount of inert prodrug to the subject. In this way, the inert prodrug is brought into contact with the immobilized enzymes and thereby converted into an active drug at the target cells or tissue.

In another aspect, the present invention provides a method of treating a disease in an individual, said method comprising localizing a substrate comprising one or more immobilized enzymes capable of converting an inert prodrug into an active drug agent to a target site for treatment, and systemically administering an efficient amount of said inert prodrug to said individual, thereby bringing said prodrug into contact with said one or more enzymes and thereby converting said inert prodrug into an active drug at the target site for treatment.

The invention also provides substrates, kits and products such as stents, such as vascular grafts, for use in medical treatment, as described herein below.

The methods of the invention can be used to administrate active drugs to any tissue or cell type, which is adhering and/or adjacent to a substrate of the invention. The methods are not restricted to any specific cells or tissue types. In a preferred embodiment, the methods are used to administer one or more active drugs to any malignant cell type, cancer cells, and/or tumour tissue. However, the methods may also be used to provide active drug agents to cells in in vitro cell cultures, where cells, such as a subset of the cultured cells, adhere to or are adjacent to a substrate of the present invention.

Modulating Enzymes, Prodrugs and Active Drugs

The methods, substrates, kits, devices and uses of the present invention can be used for simple sustained-release drug delivery systems. However, the methods, substrates, kits, devices and uses also allows spatio-temporal modulation by varying the time intervals when the prodrug in provided, the amount of immobilized prodrug and/or the spatial localization of immobilized enzyme in the substrate. A preferred embodiment of the methods of the invention comprises that the inert prodrug is provided temporally only when the action of the active drug agent is required to be administered to said cells adhering and/or adjacent to said substrate.

The substrates of the invention are not limited to those comprising only one enzyme capable of converting an inert prodrug into an active drug agent. In fact, the substrates may comprise a plurality of different such enzymes, which are chosen based on the specific application of the substrate. In this way, the substrate may comprise different enzymes, each of which is capable of converting a specific inert prodrug, thereby enabling a plurality of different prodrugs to be activated in the same substrate. Thus, the immobilization of two or more different enzymes within or at the substrate, could allow the conversion of two or more different types of prodrugs. In this way, a drug delivery system can be established, wherein a plurality of different active drugs can be produced in situ at the substrate and delivered to adhered and/or adjacent cells both simultaneously and/or independently from each other. The inclusion of multiple enzymes provides additional possibilities for modulating drug activation process. Different enzymes would allow the temporal modulation of drug delivery, because different prodrugs can be administered at different intervals, so that a first prodrug can administered first, which would lead to its conversion in the substrate. When the action of the first drug is not required anymore, the administration of the first prodrug is terminated, and then a second prodrug can be administrated later with subsequent later conversion and delivery of the corresponding second drug. Thus, the addition of a plurality of prodrug converting enzymes in the substrate would allow the temporal modulation of the administration of different active drug agents to the target cells or tissue. Thus, the substrates comprise at least one immobilized enzyme capable of converting an inert prodrug into an active drug agent, and the substrate may comprise 2, 3, 4, 5, 6, 7, 8, 9, 10 or even more different such enzymes.

In a particular embodiment, the methods and uses of the invention comprise spatial modulation of the drug administration by organizing the positioning of the enzymes immobilized in the substrate. In this way, a first specific sector of the substrate of the invention can comprise a first specific enzyme capable of converting a first specific prodrug to active drug agent, while a second sector of the substrate comprise a second specific enzyme capable of converting a second specific prodrug to a second active drug agent. In this way, cells adhering and/or adjacent to the first sector are administered the first active drug agent while cells adhering and/or adjacent to the second sector are administered the second active drug.

Thus, in one embodiment, the substrate of the invention comprise a first specific sector comprising a first specific enzyme capable of converting a first specific prodrug to a first active drug agent, and a second specific sector comprising a second specific enzyme capable of converting a second specific prodrug to a second active drug agent. The substrate of the invention may further comprise one or more additional sectors each comprising at least one specific enzyme capable of converting at least one additional specific prodrug to active drug agent.

Also, the methods and uses of the invention are not confined to single prodrugs. Just as the substrates may comprise a plurality of enzymes, the methods may include administering multiple prodrugs. Such an approach would allow the administration of different active drug agents in the methods of the invention, because each inert prodrug is converted to a specific active drug agent. The prodrugs can be administered either simultaneously or sequentially, or one or more prodrugs could be administered simultaneously and one or more alternative prodrugs could be administered at a different time interval. Thus, the administration of a plurality of prodrugs allows a temporal variation of the active drug delivery, because different prodrugs can be administered at different intervals, thereby modulating the temporal administration of different active drugs.

One embodiment of the methods of the invention therefore comprise providing at least one inert prodrug to the substrate of the invention, and the methods may comprise providing 2, 3, 4, 5, 6, 7, 8, 9, 10 or even more different prodrugs.

The amount of active drug agent administered to cells according to the methods of the present invention correlates with the amount of enzymes immobilized in the substrate of the invention. Thus, if the substrate comprises relatively high amounts of immobilized prodrug-converting enzymes, the prodrug is converted more efficiently, and thereby relatively higher amounts of active drugs are administered to cells adhering and/or adjacent to the substrate. Conversely, if the substrate comprises relatively low amounts of immobilized prodrug-converting enzymes, the prodrug is converted less efficiently, and thereby relatively low amounts of active drugs are administered to cells adhering and/or adjacent to the substrate. In this way, the amount of active drug agent administered to cells adhering and/or adjacent to a substrate according to the methods of the present invention may be modulated by adjusting or changing the relative amount of enzyme immobilized in the substrate of the invention. So in one embodiment of the methods of the invention, the relative amount of active drug administered is modulated by a corresponding change in the relative amount of enzyme immobilized in the substrate.

The amount of active drug agent administered to cells according to the methods of the present invention also correlates with the level of prodrug provided to the substrate. If relatively high amounts of prodrugs are provided, correlating higher amounts of active drug is produced within the substrate, and thereby the amounts of active drug agents administered to adhering and/or adjacent cells are increased correspondingly. Conversely, when relatively low amounts of prodrugs are provided, correlating lower amounts of active drugs are produced within the substrate, and thereby the amounts of active drug agents administered to adhering and/or adjacent cells are decreased correspondingly. Thus, in one embodiment of the methods of the invention, the relative amount of active drug administered is modulated by a corresponding change in the relative amount of inert prodrug provided to the substrate. This also means that in one preferred embodiment of the methods of the invention, the amount of the one or more provided inert prodrugs reflects the level of active drug agent to be administered to said cells adhering and/or adjacent to said substrate.

Substrate

The present invention relates to methods of administrating active drug agents, and methods of treating diseases, which comprise the use of a substrate comprising one or more immobilized enzymes, which are capable of converting an inert prodrug into an active drug agent. The invention also relates to such substrates for use in medicine, such as for use in treating cardiovascular disorders or cancers. The invention is not restricted to any specific type of substrate; any substrate to which a prodrug-converting enzyme can be immobilized may be used. The enzyme may be immobilized within the substrate and/or it may be attached to the surface of the substrate. The substrate may itself be a surface, however in certain embodiments, the substrate of the invention is a structure, such as a micro or nanostructured entity or an implantable device and/or a stent or graft, as described below. In particular, the substrate may be a surface/coating on a micro or nanostructured entity or an implantable device and/or a stent or graft.

The substrate of the invention is in one embodiment a solid material, or a substantially solid material. In one embodiment, the substrate is a gel. A gel is a solid, jelly-like material that can have properties ranging from soft and weak to hard and tough. Gels are substantially dilute cross-linked system, and there is no flow of liquid components, when the gel is in the steady-state. By weight, however, gels are mostly liquid, yet they behave like solids due to a three-dimensional cross-linked network within the liquid. It is the crosslinks within the fluid that give a gel its structure (hardness) and contribute to stickiness. In this way gels are a dispersion of molecules of a liquid within a solid in which the solid is the continuous phase and the liquid is the discontinuous phase. The structure of a gel makes it a suitable substrate for immobilizing prodrug-converting enzymes of the present invention, because the enzyme can be easily incorporated in the gel structure by standard methods, while still retaining its catalytic activity to convert inert prodrugs to active drug agents. Also, even though there is no flow of the components of a gel, a gel can still be readily diffusible, which means than molecules such as a prodrug of the invention can enter the gel and access enzymes immobilized within the gel. In this way, prodrugs provided to such a gel are converted to active drug agents in the gel, and such drug agents then easily travel by diffusion through the gel and contact the cells adhering to and/or adjacent to the gel substrate, thereby exposing these adhering and/or adjacent cells to active drug agent produced in situ within the gel.

In one embodiment, the substrate is a hydrogel. A hydrogel (also called aquagel) is a network of hydrophilic polymer chains. Hydrogels are highly absorbent natural or synthetic polymers, and can contain over 99.9% water. Hydrogels also possess a degree of flexibility very similar to natural tissue, due to their significant water content. Hydrogels therefore represent specifically suitable substrates of the present invention, in particular for use as scaffolds in tissue engineering, for use in in vitro cell culture administration of active drug agents, and/or when used as any type of implant, such as cardiovascular stents or vascular grafts or bowel stents.

In one more specific embodiment, the substrate is a polyvinyl alcohol (PVA) hydrogel. However, the substrate may also be any type of polymer, polymer thin film or porous solid matrice. Thus, in one embodiment, the substrate is selected from the group consisting of polymers, polymer thin films, and porous solid matrices. The substrate may also be selected from any suitable composition, for example from the non-limiting group consisting of Poly-N-isopropylacrylamide (pNIPAAM), Polyethylene glycol (PEG), and Alginate.

Poly(N-isopropylacrylamide) (abbreviated herein as PNIPAAM, but also known as PNIPA, PNIPAA or PNIPAm) is a temperature-responsive polymer. It forms a three-dimensional hydrogel when crosslinked with N,N′-methylene-bis-acrylamide (MBAm) or N,N′-cystamine-bis-acrylamide (CBAm). When heated in water above 32° C., it undergoes a reversible lower critical solution temperature phase transition from a swollen hydrated state to a shrunken dehydrated state, losing about 90% of its mass. In dilute solution, it undergoes a corresponding coil-to-globule transition at similar conditions). Since PNIPAAM expels its liquid contents at a temperature near that of the human body, PNIPAAM has been proposed by many researchers for possible applications in tissue engineering and controlled drug delivery. The renal glomerular filtration cut-off of copolymers of PNIPAAM is situated around 32,000 g/mol.

In another embodiment, the substrate is a layer-by-layer coating. Thus, the substrate is in one embodiment a material having a surface comprising sequential polymer deposition (layer-by-layer coating). In a preferred embodiment, such surface comprises sequential layers of polymer pairs selected from the group consisting of poly(L-lysine) (PLL)/Alginate, PLL/poly(L-glutamic acid) (PGA), thiol-modified poly(methacrylic acid) (PMAsh)/poly(N-vinylpyrrolidone) (PVP) and poly(allylamine hydrochloride) (PAA)/poly(acrylic acid) (PAH). Thus, the layer-by-layer coating is in a preferred embodiment selected from alternating layers of the following polymer pairs: poly(L-lysine) (PLL)/Alginate, PLL/poly(L-glutamic acid) (PGA), thiol-modified poly(methacrylic acid) (PMAsh)/poly(N-vinylpyrrolidone) (PVP) or poly(allylamine hydrochloride) (PAA)/poly(acrylic acid) (PAH).

The substrate of the above-mentioned aspects could be part of an implantable device, such as a stent, such as a vascular graft, or could be a stent, or any other implantable device, or a scaffold for tissue engineering. In particular, the substrate may be used as surface coating of such implantable devices.

Enzymes and Prodrugs

The methods and uses of the present invention involve one or more inert prodrugs, which can be converted to active drug agents by an enzyme. The prior art comprise numerous examples of prodrugs and enzymes, which are suitable for use in the methods, substrates and uses of the present invention. Examples of suitable prodrugs and enzymes are provided for example in Kratz et al., ChemMedChem 2008, 3, 20-53.

Prodrugs and Active Drug Agents

Prodrugs are chemicals that are inert even at relatively high doses, but can be converted to active agents the target for their action. For example, the prodrug can be a benign agent, which is converted to a toxic agent at the target, and in this way, the prodrug is benign to the organism until converted to an active toxic species. The specific activation of a prodrug is the result of an enzymatic conversion of the prodrug. For specific spatial activation of the prodrug, the activating enzyme should be either unique to the target site or present at higher concentrations at the target site. This is achieved in the present invention by immobilizing the activating enzyme in a substrate located at the site of treatment. In this way, only cells adhering to the substrate comprising the prodrug-converting enzyme is subject to the toxic and/or regulatory effects of the active drug agent.

A drug of the present invention is not necessarily a therapeutic drug or a cytotoxic drug. The term “drug”, as used herein, comprises any active agent, which can target a cell and exert an effect on that cell. A drug may thus, be any regulatory agent. Thus, the term drug also comprises for example hormones and/or other regulatory agents. Non-limiting drug agents of the present invention is a growth hormone, a cytotoxic drug agent, an anti-inflammatory drug agent, an antiviral drug agent, or an anti-platelet aggregation drug agent, such as clopidogrel. The invention also encompasses imaging methods, wherein the active drug agent is an imaging agent. In this case the prodrug is an inert (non-visible) imaging agent, which requires activation by the immobilized enzyme(s) in the substrate of the invention for visualization. Alternatively, the prodrug imaging agent and activated drug imaging agent can be distinguished by different imaging methods are visualization protocols; for example at different wavelengths. In a preferred embodiment, the drug is a cytotoxic agent, such as an agent suitable for cancer therapy.

In one embodiment of the methods, substrates, devices, kits and uses of the invention, the prodrug is a glucuronide-conjugated prodrug.

Examples of suitable drug agents of the present invention are Benzoic acid mustard or Doxorubicin, Methotrexate, 5-Fluorouracil, Nitrogen mustards, Paclitaxel or Camptothecin, such as SN-38. In one preferred embodiment, the drug agent is Nitric oxide (NO).

In one example, the prodrug is b-gal-NONOate, and the drug is nitric oxide (NO).

Thus, in one embodiment, the present invention relates to a method of administering nitric oxide to a subject in need thereof, in particular a human subject suffering from a cardiovascular disorder, said method comprising inserting an implantable device, such as a vascular stent or a vascular graft, comprising a substrate comprising one or more immobilized enzymes capable of converting an inert prodrug into an active drug agent into a blood vessel, and administering at least one inert NO prodrug, such as β-gal-NONOate, which is converted into NO.

In another embodiment, the drug is an anti-inflammatory drug agent. In particular, the drug agent may be a steroid drug, such as a glucocorticoid steroid drug, including both natural and synthetic forms. In a preferred embodiment, the drug agent is dexamethasone, and the clinical condition is inflammation and/or an auto-immune disorder.

In another embodiment, the drug is Curcumin, and the clinical condition is inflammation and/or cancer. In another embodiment, the drug is a camptothecin drug, such as SN38, and the clinical condition is cancer.

Enzymes

In the methods, substrates, devices, kits and uses of the invention, the substrate comprises one or more immobilized enzymes. In particular, the enzymes of the present invention are not generally linked to an antibody or a fragment thereof. Several well-known and suitable and applicable enzymes exist. Non-limiting examples of an enzyme of the present invention is glycosidase, beta-lactamase and/or glucuronidase. Thus, in preferred embodiments of the methods, substrates and uses of the invention, the one or more immobilized enzymes are selected from the group consisting of glucuronidase, glycosidase and beta-lactamase. In a particular preferred embodiment, the enzyme is β-glucuronidase.

Other examples enzymes suitable for immobilization in a substrate of the present invention are Carboxypeptidase G2, Carboxypeptidase A, Aminopeptidase, Glucuronidase, β-Lactamase, and/or Catalytic antibodies.

Enzyme-Prodrug Pairs

A number of suitable enzyme/prodrug pairs are known in the art. Generally, in the choice of the appropriate enzyme/prodrug combination, priority should be given to the enzyme. Suitable prodrugs can usually be designed for almost any enzyme specificity. Optimally, the enzyme should have high catalytic activity under physiological conditions, and fast and efficient prodrug activation even at low concentrations of the substrate (high _{Kcat} and low Kₘ), without dependence on further catalysis by other enzymes. However, less optimal enzymes are also usable. Thus, also enzymes, which are less efficient in terms of _{Kcat} and Kₘ are applicable. Preferably, the Kₘ of the enzymes of the invention is less than 20 μM, such as less than 19, 18, 17, 16, or 15 μM, such as less than 14, 13, 12, or 11 μM, and preferably less than 10 μm, such as less than 9, 8, 7, 6, 5, 4, 3, 2 or 1 μM. In one embodiment, the Kₘ of the enzymes of the invention is 1-20 μM, such as 1-15, such as 2-15, such as 5-15, such as 5-10 μM, or is 1-10 μM. The enzyme itself should generally not lead to cytotoxic effects. The reaction pathway of the chosen enzyme should also be different from any endogenous enzyme, in order to avoid cytotoxic activation of the prodrug in normal tissues. Thus, for applications in human beings, the selected enzyme is preferably not be a human enzyme, if this would imply a risk that the prodrug is converted in other localizations than at the site of the substrate with immobilized prodrug. However, enzymes of human origin may be advantageous in specific applications, for example in order to avoid complications of acquired immunity, in particular after prolonged prodrug administration. Also for in vitro applications, i.e. in cell culture, applications, enzymes of human origin could be used.

The selected prodrug should be chemically stable under physiological conditions and have suitable pharmacological and pharmacokinetic properties. Depending on the choice of substrate, the prodrug should be freely diffusible throughout the substrate matrix. In particular, when the enzyme is embedded within the substrate, the prodrug should be freely diffusible throughout matrix in order to reach the enzyme, and the converted active drug should also be freely diffusible in the matrix, so it can contact the cells adhering to the substrate.

For significant therapeutic gain, the released drug should be at least 2-fold, such as at least 3-fold, at least 4-fold, at least 5-fold, at least 6-fold at least 7-fold, at least 8-fold at least 9-fold, at least 10-fold more active, such as more toxic, than the prodrug, and more preferably at least 20-fold, such as at least 30-fold, at least 40-fold, at least 50-fold, at least 60-fold at least 70-fold, at least 80-fold at least 90-fold, such as 100 fold more active, such as more toxic, than the prodrug. The active, such as toxic, agent should also have a half-life that allows diffusion to the adhering cells. However, the active, such as toxic, agent should not remain constantly active with the risk that any active drug may escape into the circulation or to distant neighbouring cells. Moreover, in certain embodiment, the induced cellular effect, such as cytotoxic effect, should be cell cycle phase- or proliferation-independent, in order to be able to affect, such as kill, a wide range of cells, for example tumour cell populations.

Not all of the known enzyme-prodrug combinations possess the preferred characteristics mentioned above. However, it is also understood that the enzymes and prodrugs of the present invention need not fulfil any or all of the properties mentioned above. However, these properties could be taken into account when choosing enzyme/prodrugs for a specific application.

Examples of suitable enzyme/prodrugs combinations are herpes simplex virus thymidine kinase/ganciclovir; cytosine deaminase/5-fluorocytosine; nitroreductase/cb1954; cytochrome p450/cyclophosphamide; carboxypeptidase g2/cmda; and horseradish peroxidase/indole-3-acetic acid.

Other examples of enzymes/drug pairs are:

Carboxypeptidase G2/Benzoic acid mustard or Doxorubicin;

Carboxypeptidase A/Methotrexate;

Aminopeptidase/Methotrexate;

β-Glucuronidase/Doxorubicin, Camptothecin or 5-Fluorouracil;

β-Lactamase/Nitrogen mustards, Doxorubicin, or Paclitaxel;

Catalytic antibodies/Camptothecin

Applications

The methods, substrates and other products mentioned herein can be used for a large number of applications, both medical and non-medical applications. The methods, uses, substrates, kits, devices, stents and other products can be used in biotechnology for e.g. in vitro culturing of cells, for example for culturing of artificial tissues, wherein a subset of cells are targeted with an active drug agent without affected the remaining cells of the culture. This would allow differential differentiation of cells within a multicellular culture, which could for example be applied in in vitro tissue engineering.

Medical Use

In a particular aspect, the present invention also relates to the medical use of the methods, substrates, kits, devices, stents and other products mentioned herein. For example, the invention in one aspect provides a substrate comprising one or more immobilized enzymes capable of converting an inert prodrug into an active drug agent for use in medicine, in particular for the treatment of cancer or cardiovascular disorders. Also, the invention provides in one aspect, a method of administering an active drug agent to cells adhering and/or adjacent to a substrate, said substrate comprising one or more immobilized enzymes, which are capable of converting an inert prodrug into an active drug agent, said method comprising providing at least one inactive prodrug to said substrate. Such a method could comprise the steps of providing said substrate comprising one or more immobilized enzymes, which are capable of converting an inert prodrug into an active drug agent, allowing cells to contact said substrate, and providing one or more prodrugs to said substrate. By contacting said substrate, the one or more prodrugs will be converted into active drug agent at the site of the substrate, said active drug agents consequently contacting said cells adhering and/or adjacent to a substrate.

In a further aspect, the invention provides a method of administering an active drug agent to cells adhering and/or adjacent to a substrate by in situ production of the drug within said substrate, wherein said substrate comprises one or more immobilized enzymes, which are capable of converting an inert prodrug into an active drug agent. Such a method is for example an in vitro method, where active drug agent is provided to cells adhering to the substrate. This method of administering active drug agent could be used in in vitro tissue engineering, for example, in a scaffold for tissue engineering.

In addition, the invention relates to a method of treating a disease in an individual, said method comprising localizing a substrate comprising one or more immobilized enzymes capable of converting an inert prodrug into an active drug agent to a target site for treatment, and systemically administering an efficient amount of said inert prodrug to said individual, thereby bringing said prodrug into contact with said one or more enzymes and thereby converting said inert prodrug into an active drug at the target site for treatment.

The invention also provides in one aspect an implantable device consisting of or comprising a substrate of the present invention. In particular, the invention provides an implantable device consisting of or comprising a substrate of the present invention for use in the treatment of cancer. The device can be inserted into a tissue, which is a target for treatment/administration of the drug, and the inert prodrug can be provided by systemic administration to an organism, by a method of the present invention. The tissue is for example a cancer tissue. Thus, the invention provides a method for the treatment of cancer, wherein an implantable device consisting of or comprising a substrate of the present invention is inserted in the body of a patient at the site of the cancer tissue, and administering an inert prodrug, which is converted to an active anticancer drug at the site of the implantable device, i.e. at the site of the cancer tissue. This method may be combined with one or more additional treatments for cancer, for example radiation therapy or chemotherapy.

The methods and substrates of the invention are claimed for use in the treatment of any clinical condition, which would benefit from the time and dose dependent drug delivery at a specific target site. In a particular embodiment, the methods and substrates of the invention are claimed for use in the treatment of cardiovascular disorders. In the treatment of vascular disorders, a substrate of the invention, which comprise a prodrug as described elsewhere herein could be inserted within the vascular system of a patient, and from this position used to administer drugs into the vascular system in a site-specific, dose-specific and/or time-dependent manner. In particular, the substrate of the invention can in this case be formed as a vascular stent, such as a vascular graft, and/or the active drug agent could be an anti-coagulating drug agent, which would inhibit blood coagulation and thereby reduce the risk of blood clotting in the vascular system. This could be relevant at positions in the vascular system, where there might be increased risk of thrombosis.

Thus, in one aspect, the invention relates to a method of treating atherosclerosis, said method comprising inserting a vascular stent, such as a vascular graft, comprising a substrate comprising one or more immobilized enzymes capable of converting an inert prodrug into an active drug agent into a blood vessel, and administering an inert prodrug, which is converted into an active anti-coagulating drug agent.

In another embodiment, the methods and substrates are claimed for use in the treatment of cancer tumours. In this case the substrate of the invention may be inserted within a site of a tumour, for example in the tissue, from which a tumour has been surgically removed. In this case, anti-cancer drug agents can be administered by SMEPT, where inert anticancer prodrugs are provided to the substrate by systemic administration of the prodrug to the patient, and then the prodrug will translocate to the tissue, where the substrate has been introduced, and in this location be converted to active drug, such as an active chemotherapeutic drug or other cytotoxic compound.

Implantable Devices

In one aspect, the present invention relates to an implantable device comprising a substrate of the invention, i.e. a substrate comprising one or more immobilized enzymes capable of converting an inert prodrug into an active drug agent. The implantable device can be any device, from which the release of active drug agent could be useful. In a preferred embodiment, the substrate is integrated in a stent, such as a vascular graft.

Thus, in one embodiment, the substrate of the present invention is integrated in, or constitutes a stent, such as a vascular graft, suitable for insertion in a human or animal body. A stent is any device which is inserted into an internal duct in order to expand the duct to prevent or alleviate a blockage. Most prominently, stents are used to expand blood vessel in order to avoid blockage. The substrate of the present invention is in one embodiment part of a stent or is a stent, or a vascular graft, an implantable device, or a scaffold for tissue engineering.

Thus, in one aspect, the present invention relates to a stent, such as a vascular graft, comprising a substrate comprising one or more immobilized enzymes capable of converting an inert prodrug into an active drug agent. The invention also relates to a substrate comprising one or more immobilized enzymes capable of converting an inert prodrug into an active drug agent for use as a stent, such as a vascular graft. Also, the invention relates to a stent, such as a vascular graft, comprising a substrate comprising one or more immobilized enzymes capable of converting an inert prodrug into an active drug agent.

The stent of the invention is for example a cardiovascular stent, a bowel stent, a ureteral stents, a prostatic stent, an esophageal stent, a biliary stent, or a stent graft or a vascular graft.

When used as a stent, such as a vascular graft, the substrate of the present invention is preferably chosen from stable materials, which remain in the body permanently or until removed through surgical intervention. In one embodiment, when used as a stent, such as a vascular graft, the substrate of the present invention can be selected from biodegradable or bioresorbable materials. In this case, the stent is degraded or resorbed after a certain time, when the stent and/or the drug released from the stent is no longer required.

Kit

One aspect of the present invention relates to a kit comprising a substrate comprising one or more immobilized enzymes capable of converting an inert prodrug into an active drug agent, and said inert prodrug, where the substrate, enzyme, prodrug and active drug agent are as defined herein above.

The kit may comprise any additional components necessary or suitable for carrying out any methods or uses of the present invention. In one embodiment, the kit comprises an instruction manual for using the kit, such as instructions in performing any method or use of the present invention.

In one embodiment, the kit comprises a stent of the invention, as defined elsewhere herein, for example a cardiovascular stent, a bowel stent, a ureteral stents, a prostatic stent, an esophageal stent, a biliary stent, or a stent graft or a vascular graft.

### EXAMPLES

### Example 1

Substrate Mediated Enzyme Prodrug Therapy (SMEPT)

Controlled drug delivery mediated by implantable devices is a paradigm with a major academic and commercial impact on tissue engineering and reconstructive surgery. However, a phenomenological limitation of drug eluting substrates in their current form is a lack of “post-implantation” control over drug release. Substrate Mediated Enzyme Prodrug Therapy (SMEPT) overcomes this using enzymes immobilized within a substrate, such as a cell culture substrate, to achieve in situ conversion of benign, inert prodrug(s) into active drug agents, such as therapeutics, with subsequent delivery to adhering cells or adjacent tissues. The present example demonstrates several independent arms of control over the rate and the amount of generated product made available “postimplantation”, demonstrate that SMEPT is devoid of “burst release”, achieve generation of reporter cargo in biomedically relevant concentrations, and present time- and dose-dependent cellular internalization of model cargo achieved through SMEPT. This highly adaptable concept overcomes a fundamental limitation of drug delivery engineered into implants and tissue engineering matrices.

The present example disclose SMEPT implemented in a microstructured (μS), micrometer-thick surface adhered physical hydrogels based on a polymer with decades of biomedical prominence, poly(vinyl alcohol), PVA. The choice of the substrate was driven by the following criteria: PVA hydrogels are among the most well characterized materials in tissue engineering yet suffer from poor control over drug immobilization and release and would therefore tremendously benefit from engineered opportunities in controlled drug release. Further, μS and micro-patterned substrates are pivotal in culture and, predominantly, co-culture of mammalian cells towards 2D and 3D reconstruction of organs and tissues. Finally, surface-adhered substrates have recently gained recognition as powerful tools in surface mediated drug delivery for e.g. prevention of restenosis and facilitated acceptance of implants. Together with serving as appropriate model substrate for the above-mentioned applications, surface adhered nature of the substrates allows using a host of techniques for visualization and characterization of the substrate.

This embodiment of SMEPT capitalizes on an inherent feature of hydrogel materials and PVA hydrogels in particular, namely a high permeability towards small cargo. When immobilized within cell adhesive hydrogels, an enzyme is expectedly accessible to the benign (inert), nonactive pro-drug, convert it into an active product and the latter will therefore be released in an immediate vicinity of the adhering cells. Prior knowledge on “antibody directed enzyme prodrug therapy” (ADEPT) delivers a choice of enzyme—prodrug pairs with documented biomedical relevance and successes; cf. Tietze, L. F. & Krewer, B. Antibody-Directed Enzyme Prodrug Therapy: A Promising Approach for a Selective Treatment of Cancer Based on Prodrugs and Monoclonal Antibodies. Chem Biol Drug Des 74, 205-211, doi:DOl 10.1111/j.1747-0285.2009.00856.x (2009); NiculescuDuvaz, I. & Springer, C. J. Antibody-directed enzyme prodrug therapy (ADEPT): A review. Adv Drug Deliver Rev 26, 151-172 (1997); Kratz, F., Muller, I. A., Ryppa, C. & Warnecke, A. Prodrug Strategies in Anticancer Chemotherapy. ChemMedChem 3, 20-53, doi:10.1002/cmdc.200700159 (2008). In this specific example, β-glucuronidase enzyme (β-Glu) and glucuronide prodrugs was chosen as a system with adequate prior characterization, and also being commercial available.

To ensure that the results provided in this example are readily reproducible, in all experiments and despite availability of custom made polymers, commercial polymer samples were used throughout the study. μS PVA hydrogels were assembled using PVA with molecular weight 89-98 kDa via micro-transfer molding (μTM). In brief, solution of PVA was placed between an elastic mold and a glass coverslip and pressed at finger-tight pressure for 24 h. Upon detachment, cover-slip-adhered μS PVA films were subjected to a 1 h stabilization using a coagulating salt, sodium sulfate. Pristine, non-stabilized samples dissolve upon a contact with water or aqueous buffers. In contrast, non-cryogenic physical gelation of PVA using “salting out” gives rise to well-defined, robust surface-adhered hydrogels. For protein immobilization, β-glucuronidase was mixed with PVA prior μTM through pipette assisted aspiration, and resulting solution was used in the preparation of μS PVA hydrogels according to the above protocol with no variation.

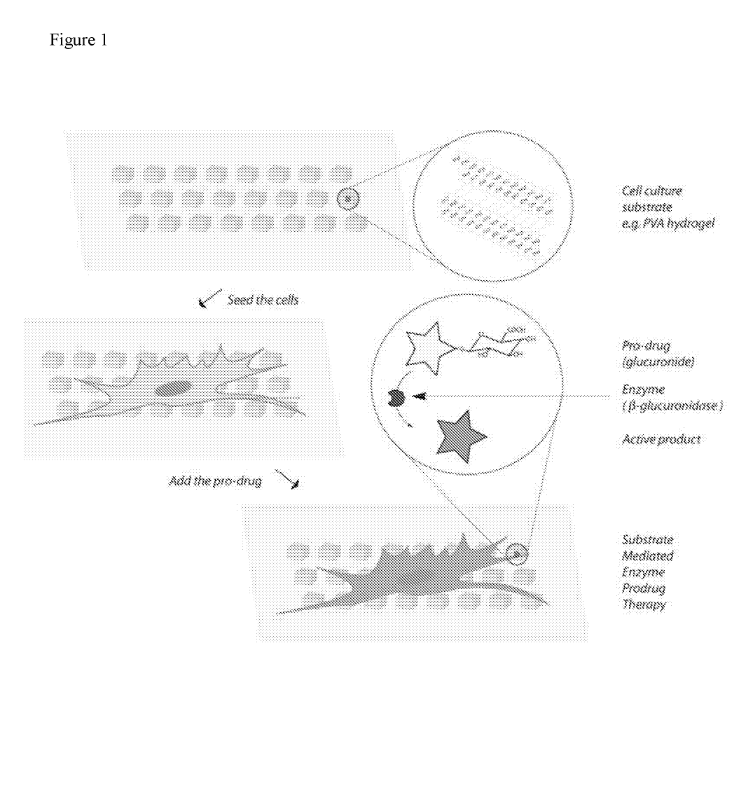

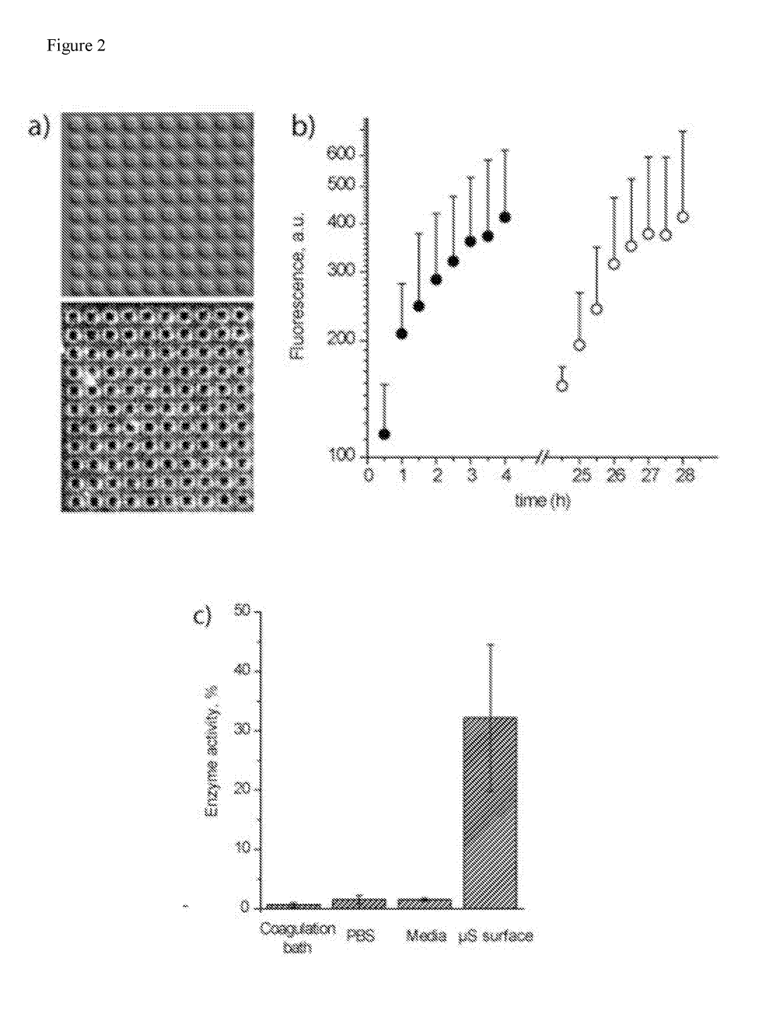

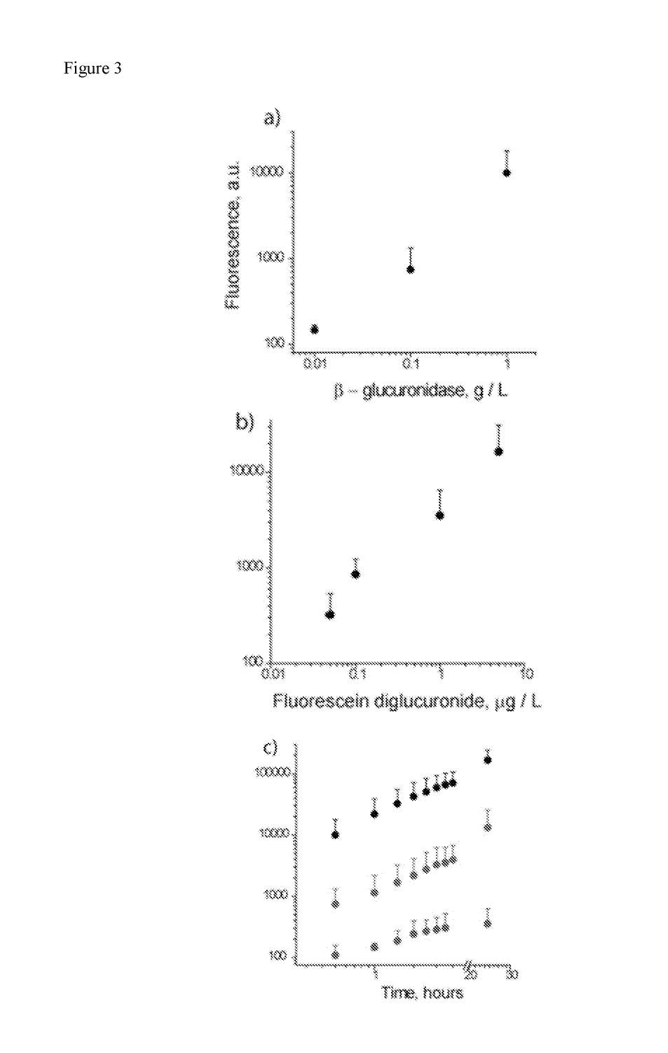

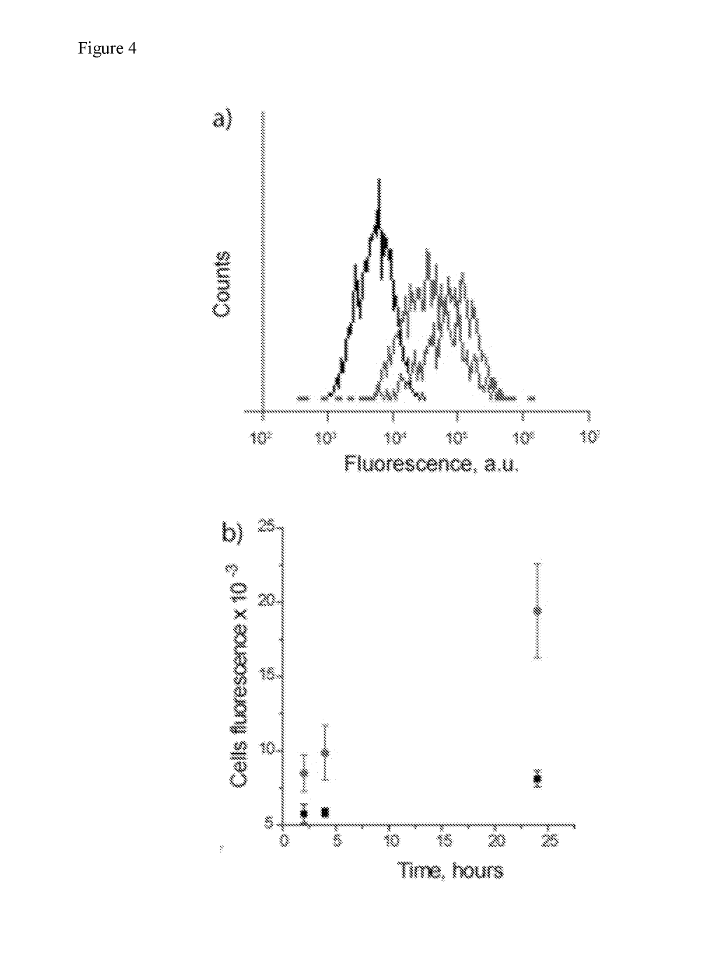

FIG. 1, a demonstrates microscopy images of the resulting μS PVA thin films visualized in a hydrated state in PBS in differential interference contrast (DIC) and fluorescence mode. In the latter case, a fluorescently labeled protein sample was used to facilitate visualization. The presence of the protein appears to have non-detectable influence on the stability of μS PVA films which remain structurally stable and robust. A preferential localization of the protein on the perimeter of the individual 2 μm-sized microstructures correlates well with the previously observed re-distribution of the PVA with these macromolecular characteristics during the process of hydrogelation, as reported in previous publications, by Zelikin and co-workers; cf. Jensen, B. E. B. et al. Poly(vinyl alcohol) Physical Hydrogels: Noncryogenic Stabilization Allows Nano- and Microscale Materials Design. Langmuir 27, 10216-10223, doi:10.1021/1a201595e (2011).

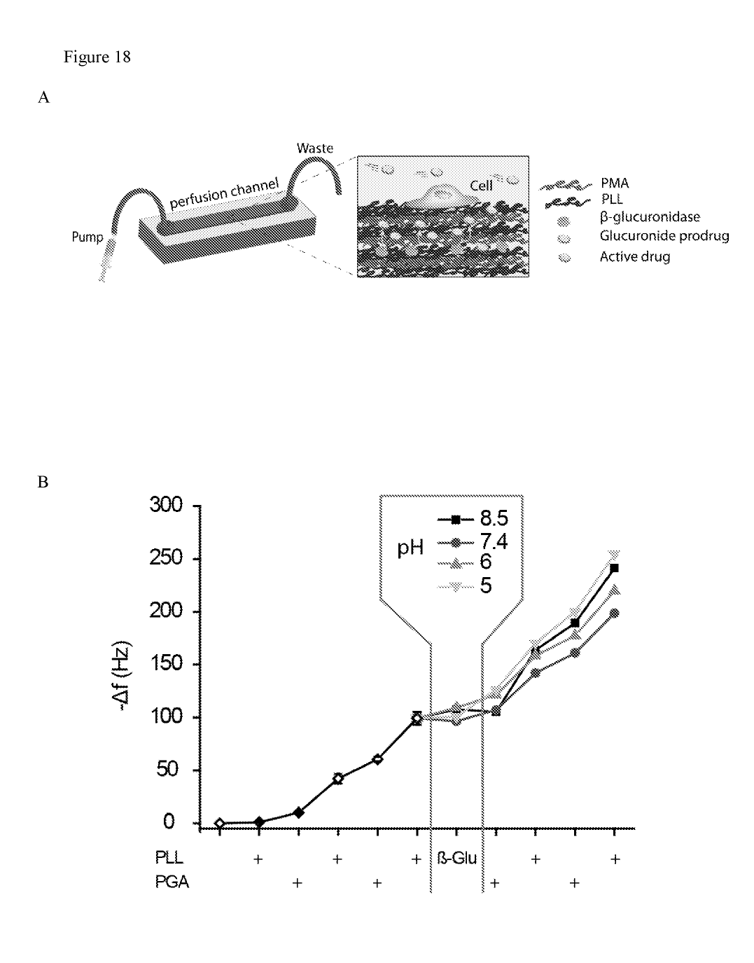

To quantify enzymatic activity, we employed a di-glucuronide derivative of fluorescein (FG), a non-fluorescent substrate, enzymatic conversion of which yields a highly fluorescent product (FIG. 18 B,C). In agreement with the proposed concept, addition of the substrate initiated a pronounced and continuous evolution of fluorescence indicative of in situ generation of the product (FIG. 18B). To verify that enzymatic conversion is accomplished within the structure of the hydrogels and not by the enzyme molecules lost from the structures into solution bulk, “coagulating salt”, PBS wash and cell culture media wash were tested alongside the μS PVA thin films in an enzymatic conversion of the prodrug into a fluorescent product, FIG. 18C. For each sample, the level of enzymatic activity is expressed relative to solution based activity of the enzyme in an amount equivalent to that used for the preparation of μS surfaces. When supplemented with a fluorogenic substrate, the tested supernatants developed minor levels of fluorescence only marginally exceeding background levels of fluorescence and significantly lower than the levels attained through a substrate conversion by the gel-embedded enzyme. These data clearly show a low level of activity of the enzyme in the solutions above the μS PVA thin films and imply that substrate conversion is predominantly achieved by the gelembedded enzyme. Note also, that in biomedically relevant media flow conditions and by analogy with ADEPT, the released enzyme will be effectively removed from the substrate vicinity further contributing to the specificity of substrate conversion achieved through SMEPT.

FIG. 1,B further demonstrates that the rates of presentation of in situ generated product obtained using identical samples and initiated 24 h apart are near identical. This graph therefore illustrates the points of advantage of SMEPT over existing tools in controlled drug release and surface mediated drug delivery (SMDD) in particular, namely the nature of the “trigger” for commencement of drug release. Typically, the moment of “implantation” (e.g. an immersion into a model medium) marks the initiation of the processes, i.e. drug release. In stark contrast, “implantation” of the enzyme-loaded PVA hydrogels is not associated with a commencement of drug release, and the latter is initiated at the moment of choice by administration of the pro-drug. Initial immersion into a test release milieu produces no active product revealing that by design, SMEPT is devoid of “burst release” phenomenon, the latter being a persistent shortcoming of drug eluting matrices and hydrogels in particular. Furthermore, this data illustrate a unique opportunity of SMEPT, namely a control over drug dosage achieved “post-implantation”. The following experiments provide further verification of this notion.

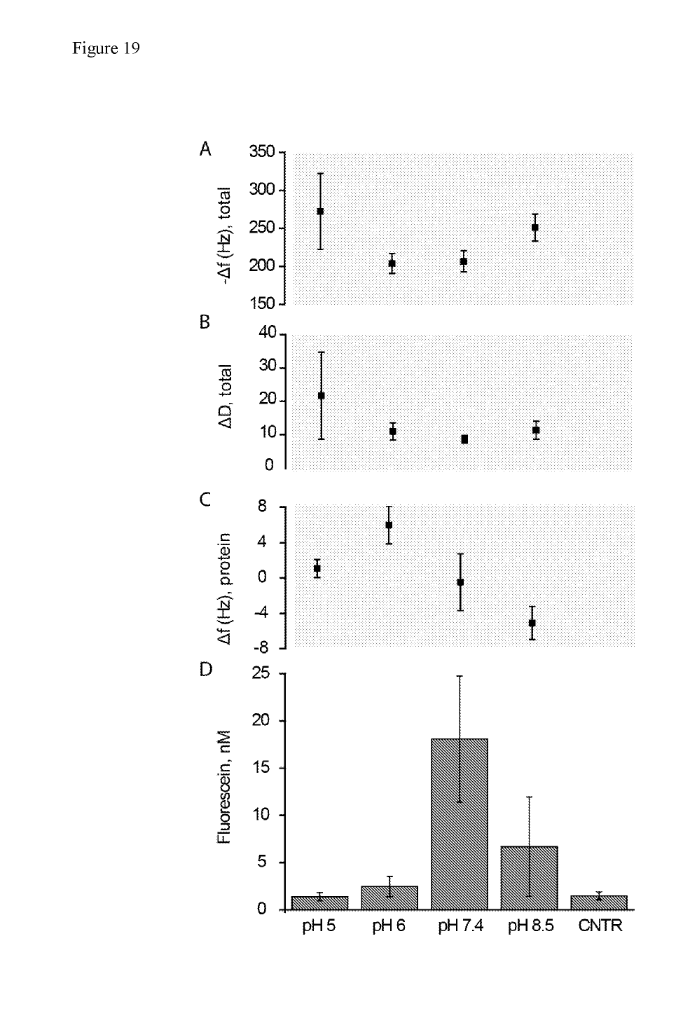

By design, the flexible and adaptable nature of SMEPT allows to control the rate of drug presentation and the overall quantity of the generated product by several independent methods. At a constant concentration of a prodrug, generation of the product can be rationally programmed by the concentration of the protein within the μS PVA thin film, FIG. 19A. Higher enzyme content achieves a faster conversion of the substrate, and the end-point analyses conducted after 30 minutes of enzymatic reaction reveal progressively higher levels of solution fluorescence attained with increased content of the enzyme in the gel. Presented data demonstrate that a single administered dose of a pro-drug can yield a judiciously chosen concentration of the active product at the site of action varied over at least 2 orders of magnitude. An envisioned biomedical utility of this arm of control relates to multiple (two or more) sites of activity of the same drug whereby the concentration at each site can be engineered individually and independently.

While a “built-in” control over drug release such as that presented above can be achieved by most successful platforms, experiments presented below demonstrate that SMEPT allows a dosing and a time-controlled presentation of an in situ generated product to be fine-tuned “post-implantation”, a feature that renders SMEPT a stand-alone paradigm in controlled drug delivery. At a given enzyme loading, the rate of drug presentation is conveniently determined by the concentration of administered pro-drug. The latter can be varied over orders of magnitude (FIG. 19B), engineered to take into account parameters of enzymatic catalysis (Km etc) for particular glucuronide pro-drugs, and yield a concentration of the generated drug to suit the particular application. The overall amount of the product is further controlled by the time of enzymatic reaction, FIG. 19C, and together, the pro-drug concentration and time of exposure allow in situ generating the product in concentrations varied over several orders of magnitude.

While the above experiments demonstrate that SMEPT is a highly flexible technique for engineering in situ generation of the product and a rate of drug release, it is also relevant to demonstrate the implementation of SMEPT for drug delivery to cultured cells. Towards this goal, hydrogels were prepared using solutions of PVA supplemented with a synthetic polypeptide, poly-L-lysine (PLL). While pristine PVA hydrogels do not support cell adhesion, PLL-containing gels have proven to be suitable substrates for cell culture. Enzyme- and PLL-containing PVA hydrogels were assembled as described above, after which time μS thin films were used as substrates for adhesion and proliferation of a model cell line, HepG2. Inasmuch as liver failure is among the leading causes of death worldwide, hepatic tissue engineering attracts increased research attention and would significantly benefit from engineered substrate mediated controlled drug release, as presented herein. Furthermore, advanced efforts in engineering of liver tissue require co-culturing of mammalian cells (e.g. hepatocytes and Kupffer cells), a challenge which is successfully addressed using μS and micro-patterned substrates. These notions together justify the use of a hepatic cell line and μS PVA hydrogels for the development of SMEPT.

Initial, internalization of a fluorescent reporter molecule produced via SMEPT and via a solution-based conversion was compared. To achieve this, the cells were seeded on μS PVA hydrogels and were allowed 24 h for attachment, after which time the media was changed to fresh media and, for solution based prodrug conversion, equivalent amount of the enzyme as that contained within μS PVA hydrogels was administered to the cultured cells. At this point, the samples were charged with fluorescein di-glucuronide to a 2.5 μg/mL concentration (3.65 μM). Upon 24 h incubation, fluorescence of the harvested cells was quantified using flow cytometry. As expected, administration of the prodrug in absence of the enzyme in solution or within the gel phase afforded no fluorescent product and the cells exhibited fluorescence identical to pristine, non-treated cells (black trace). In contrast, implementation of SMEPT afforded levels of cellular fluorescence, i.e. level of cargo internalization, comparable to that produced by a solution based administration. We note that solution based prodrug conversion is used herein only as a measure to verify protein activity and utility of SMEPT. In the human body, β-glucuronidase has a limited spread and an activity markedly lower as compared to bacterial copy used in this study. The latter notion provides for a low level of “background” prodrug conversion and contributes to specificity of drug delivery achieved with the use of β-glucuronidase and glucuronide prodrugs, as documented for ADEPT and inherited by SMEPT.

For further characterization of SMEPT, the time course of cellular internalization of the in situ generated fluorescent reporter product was followed at two concentrations of the fluorogenic pro-drug, 0.025 and 0.25 μg/mL (36.5 and 365 nM, respectively). At specified time points, fluorescence of the detached cells was quantified using flow cytometry, FIG. 19B. A pronounced increase in the fluorescence of cells was registered already at the earliest time-point, 1 h. With increased time, the cells exhibit progressively higher levels of fluorescence which provides evidence of continuous enzymatic conversion of the pro-drug and internalization of the reporter cargo. Further to this, at each time point, higher pro-drug concentration affords a higher level of fluorescence registered in the cultured cells, i.e. a greater amount of internalized cargo. Together, these two graphs present time- and dose-dependent internalization of reporter cargo and illustrate the tools of control offered by SMEPT for substrate mediated drug delivery.

Taken together, experiments presented above mark the advent of a novel research opportunity, substrate mediated enzyme prodrug therapy. The method offers an in situ generation of the therapeutic cargo in an amount and at a rate suited for particular applications achieved using an enzyme embedded within a suitable cell culture substrate. We envision that this method is highly adaptable and is not limited to the particular enzyme, prodrug and method of immobilization used in this study. SMEPT is applicable in a wide diversity of applications, specifically tissue engineering for site-specific generation of therapeutics and creation of gradients of concentration of drugs to e.g. promote cell adhesion and directed migration. From a different standpoint, PVA based implants have large clinical potentials for e.g. prevention of post-operative tissue adhesion, as embolic bodies etc. SMEPT can also be utilized for site specific delivery of anti-inflammatory and anti-viral therapeutics.

### Example 2

Engineering PVA Physical Hydrogels as Matrices for Surface Mediated Enzyme Prodrug Therapy.

This example demonstrates how PVA hydrogels can be engineered as intelligent biointerfaces and substrates for SMEPT in particular. The suitable stabilization sulfate concentration and time to generate hydrogels is determined, and assessed visually. Polymer retention in the matrix is quantitated, and protein activity within the hydrogel is monitored. In addition, the mechanical properties of the gel are quantitated via AFM force—distance measurements.

In all experiments, 12 wt % solutions of PVA with molecular weight 35 KDa were used. In this example, engineering PVA hydrogels as substrates for SMEPT is achieved through a variation of coagulation conditions used in the preparation of hydrogels. Micro-structured (μS) PVA hydrogels are prepared via p-transfer molding (μTM) with a subsequent stabilization using aqueous kosmotropic salt, sodium sulfate. Sodium sulfate in concentrations 500 mM and 1 M is equally effective in producing μS PVA hydrogels. The present example also provides a detailed investigation on the concentration of sodium sulfate as a tool for judicious design of PVA physical hydrogels, specifically in the form of surface adhered materials for SMEPT.

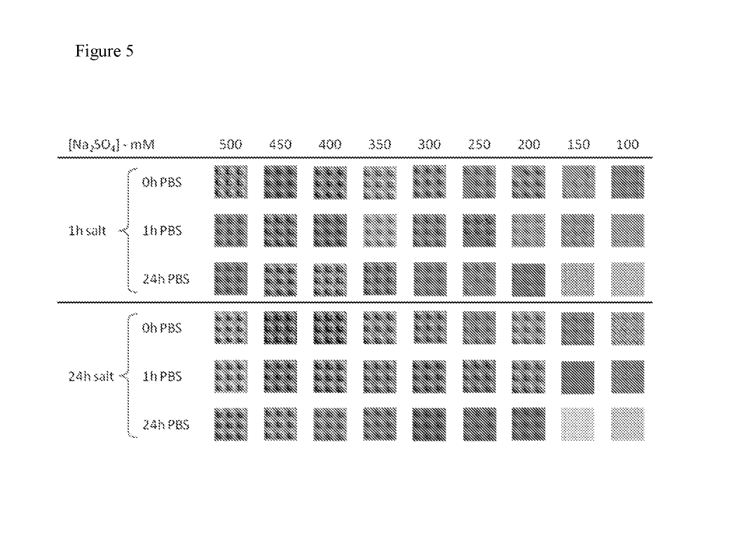

In the first experiment, μTM was followed by a stabilization of the samples is sodium sulfate baths with concentrations varied from 100 to 500 mM for 1 or 24 h. Following coagulation treatment, the samples were incubated in PBS for 1 or 24 h and visualized in a hydrated state in PBS using differential interference contrast microscopy, cf. FIG. 5. FIG. 5 shows that with extended stabilization time, as low as 200 mM salt reliably produces μS PVA hydrogels which remain stable in PBS for at least 24 h.

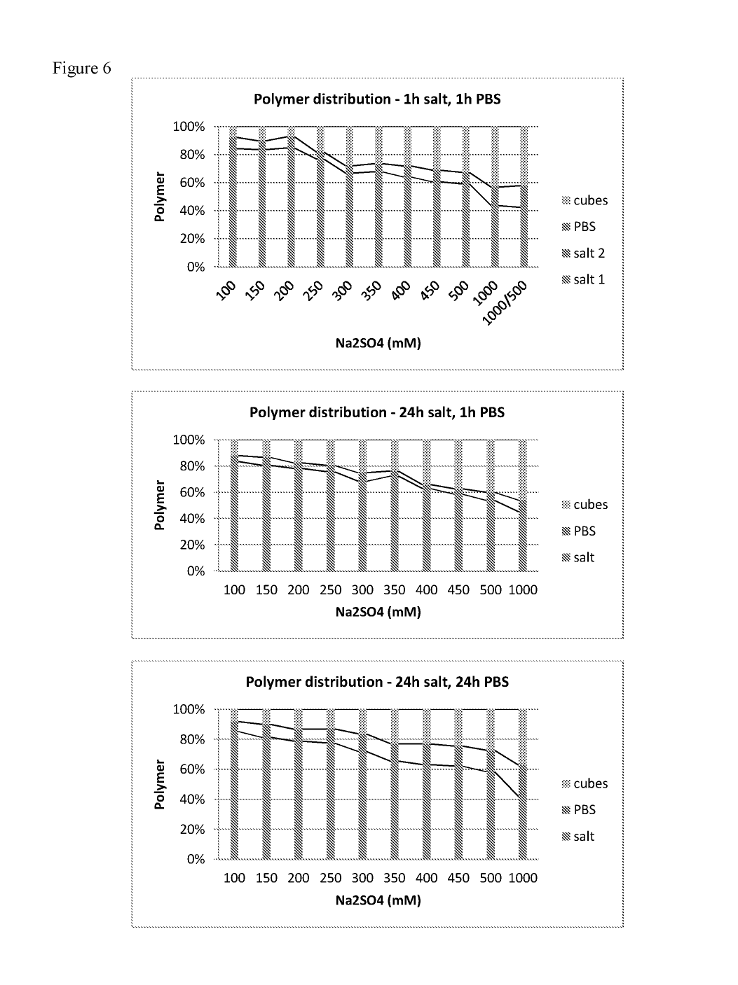

Stabilization of PVA from solution into a three dimensional hydrogel matrix is associated with a polymer loss. To quantitate this, custom made PVA with chromophore terminal groups were used to monitored polymer concentration in the stabilizing sulfate bath, in a PBS wash solution, and within the hydrogels, cf. FIG. 6. FIG. 6 shows that Polymer loss is found mostly in the sulfate bath with a relatively minor fraction lost during subsequent PBS wash. According to supporting visual observations, 100-200 mM are ineffective in retaining the polymer. Wth increased sulfate, a greater polymer fraction is retained in the structures, up to 40%. Upon incubation in PBS, some polymer loss is observed over time.

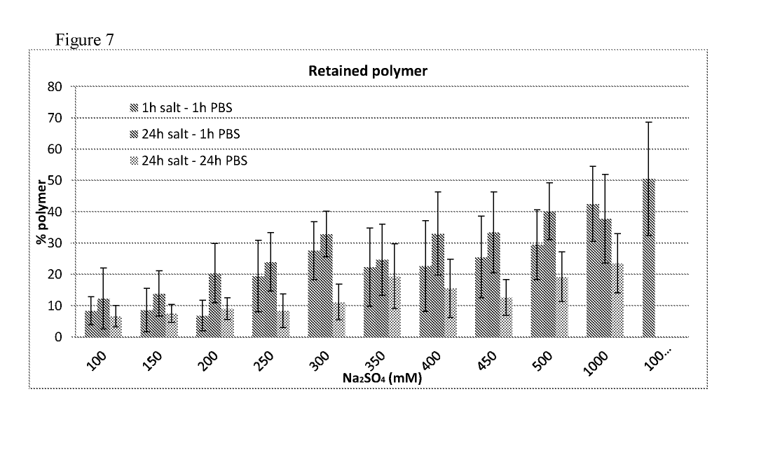

FIG. 7 shows the retention of polymer within stabilized structures using different stabilization—time and—concentration (N=3). It is seen that, as much as half the polymer is lost upon an incubation from 1 to 24 h. However, the hydrogels remain visually robust (FIG. 5) and are therefore (12 wt %×20%=2-3 t%) 2-3 wt % by mass made of polymer. Assuming that the features are homogeneous, this is 20-30 g/L.

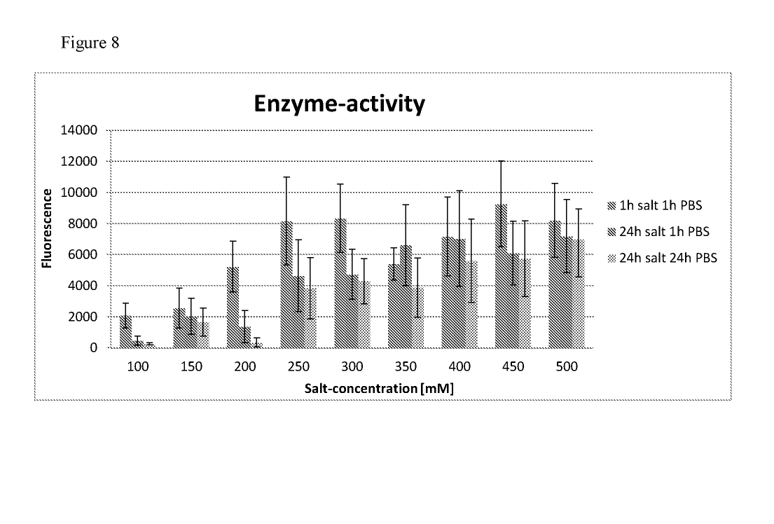

The same stabilization conditions were then used to assemble the hydrogels containing b-glucuronidase to monitor enzyme activity within μS PVA hydrogels; cf. FIG. 8. With 24 stabilization time, as low as 250 mM salt produces hydrogels with pronounced enzymatic activity. Upon incubation in PBS (1 h to 24 h) there is no significant drop in the enzyme activity. This is somewhat surprising in that we are losing half the polymer, however, it possibly implies that polymer loss is not associated with the loss of enzyme. In fact, from 250 to 500 nm, enzymatic activity does not reveal drastic changes which amount at best to 2-fold.

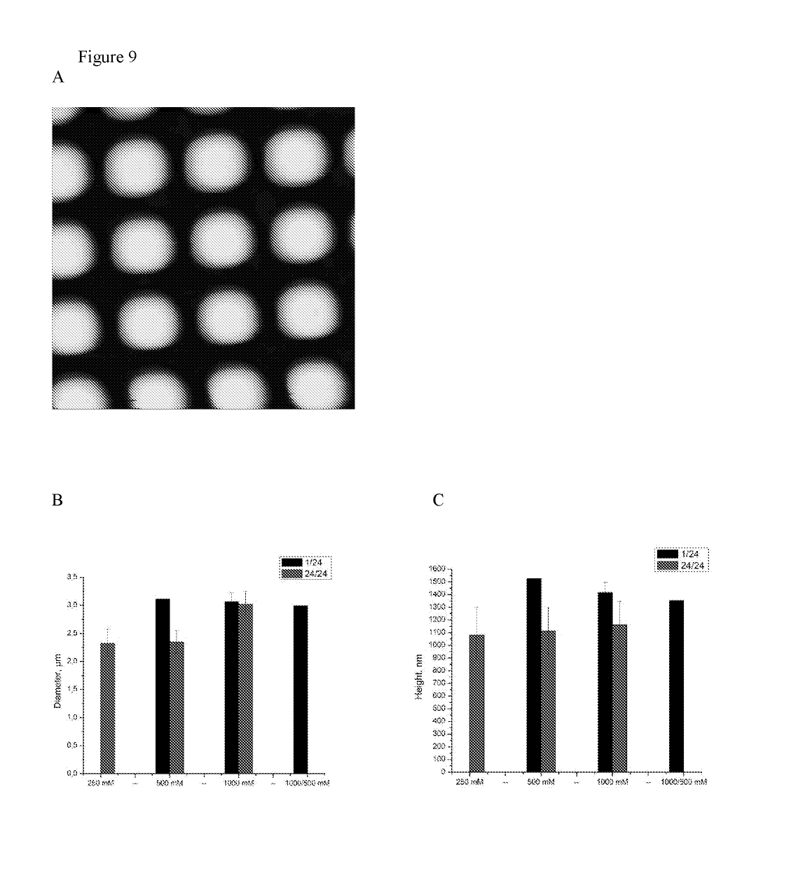

The next experiment aimed to ascertain dimensions of the topography features prepared using sodium sulfate for selected concentrations and duration of treatment; cf. FIG. 9. For the chosen concentrations, both D and H are quite similar;

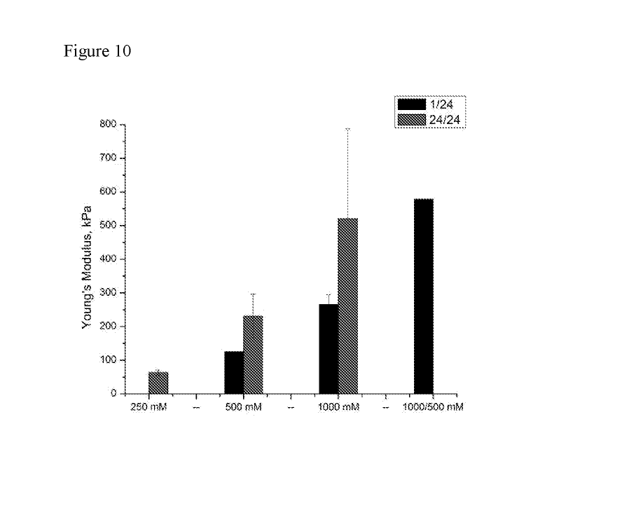

The mechanical properties of created hydrogels are of course of utmost importance. What is important from this graph shown in FIG. 10 is that the created hydrogels cover the range of Young's moduli which affects cell adhesion, proliferation and even intracellular processes. Thus, it is possible to fine tune the hydrogels mechanics using concentration of the sulfate as a powerful tool. This is highly important in that cell adhesion and proliferation are effectively controlled via substrate mechanics. However, even more important, growing evidence suggests that intracellular processes are also under a control from outside and through substrate mechanics it is possible to e.g. decouple replication and transcription. The range of Young's moduli as registered herein matches that previously reported to exert the above control.

### Example 3

PVA Hydrogels as Matrices for SMEPT.

Substrate Engineering Through Macromolecular Design.

This example demonstrates the applicability of PVA hydrogels as biointerfaces and matrices for SMEPT through variation of polymer molecular weight and concentration. Unless stated otherwise, a 500 mM sodium sulfate and 1 h stabilization time are used in this example.

In this experiment micro-structures prepared using PVA with molecular weight of 4.5, 10 and 35 kDa and concentration of 12 wt % were loaded with enzyme, salted out for 1 h with 200 μL of 0.5 M Na₂SO₄ and immersed into 1 mL PBS where aliquots were drawn after 1 h, 2 h, 4 h and 24 h and analyzed for activity with 0.25 μg/mL fluorescein di-glucuronide. This was done to see if the synthesized LMW polymers were able to retain the enzyme and its activity as the commercial polymers.

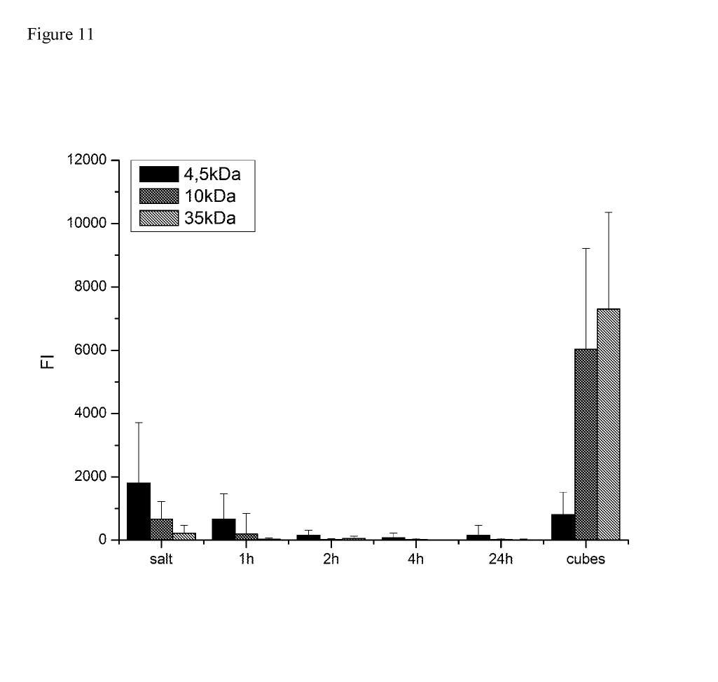

FIG. 11 introduces a method to quantify enzymatic activity during the assembly of μS PVA hydrogels as matrices for SMEPT. Enzymatic activity is quantified in the coagulation bath, subsequent PBS wash and finally within the μS PVA hydrogels. In this experiment, it is found that enzyme activity is registered mostly in the μS PVA hydrogels, and that losses are mostly detected in the stabilizing salt solution. In PBS, there appears to be a fast loss of activity of the enzyme. In contrast, within the hydrogel the enzyme remains active and ensures a well pronounced conversion of the substrate into a fluorescent product. In particular, there is a well pronounced difference between polymer samples in their molecular weight.

In the next experiment micro-structures prepared using PVA with molecular weight of 10 and 35 kDa and solutions with concentration of 2, 4, 8, 12, 16 and 20 wt % were loaded with enzyme, salted out for 1 h with 200 μL of 0,5M Na₂SO₄ and immersed into 200 μL PBS for 1 h and analyzed for activity with 0.25 μg/mL fluorescein di-glucuronide, to see if the release-profile changes with the change in wt % and MW

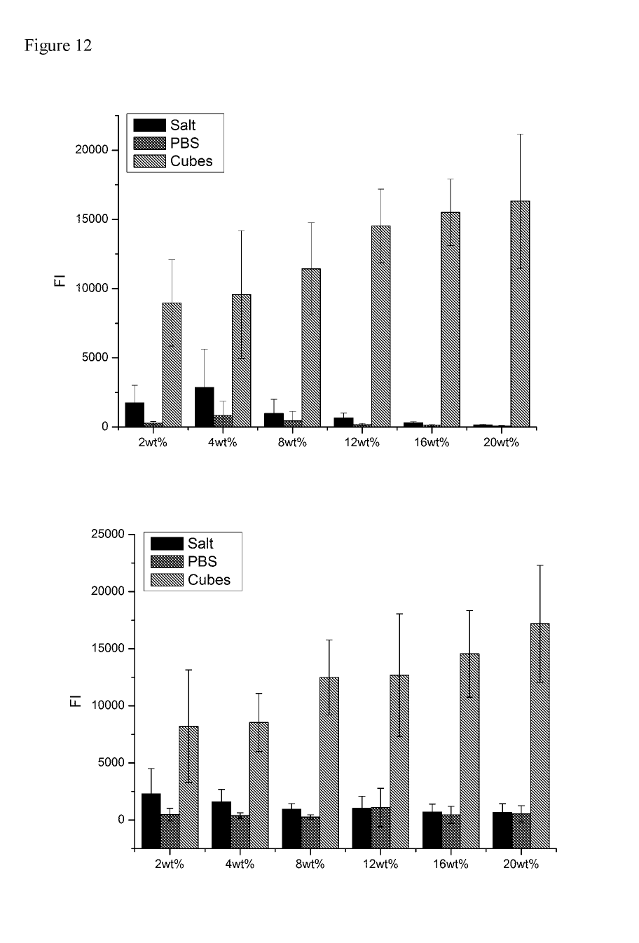

FIG. 12 shows enzymatic activity registered using μS PVA hydrogels prepared using solutions of 10 (top) and 35 (bottom) KDa PVA taken at varied concentration. With increased polymer concentration, there is a pronounced increase in the enzyme activity. Only the lowest polymer concentrations reveal a loss of enzyme into the coagulation bath. Even the lowest polymer concentrations produce hydrogels with a pronounced level of enzymatic activity.

In the next experiment, the enzyme activity was compared within the hydrogel cubes prepared using PVA with molecular weight of 4.5, 10 and 35 kDa taken at the same concentration. Activity within the 35 kDa cubes is slightly higher than the same wt % for 10 kDa in most cases. Also, the activity of the enzyme retained within the cubes of 4.5 kDa is significantly smaller than the two higher MW. So a roughly conclusion is that the higher the MW and/or wt %, the higher the retention of enzyme and thereby higher activity.

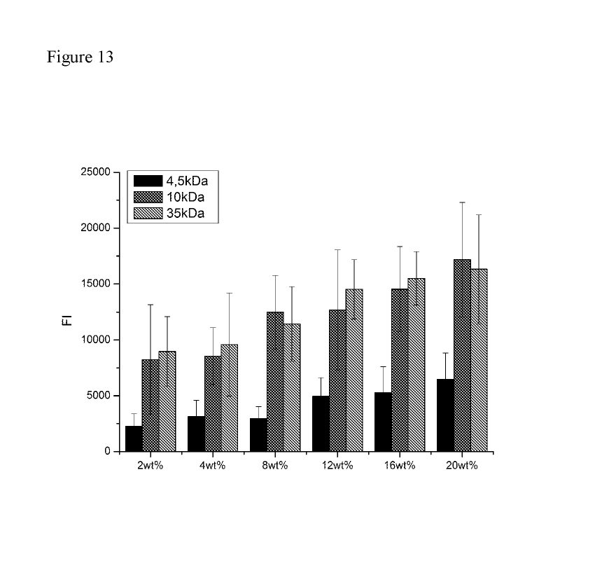

FIG. 13 shows that low molecular weight polymer has the lowest associated enzymatic activity. 10 and 35 KDa appear to be very similar with respect to enzymatic activity.

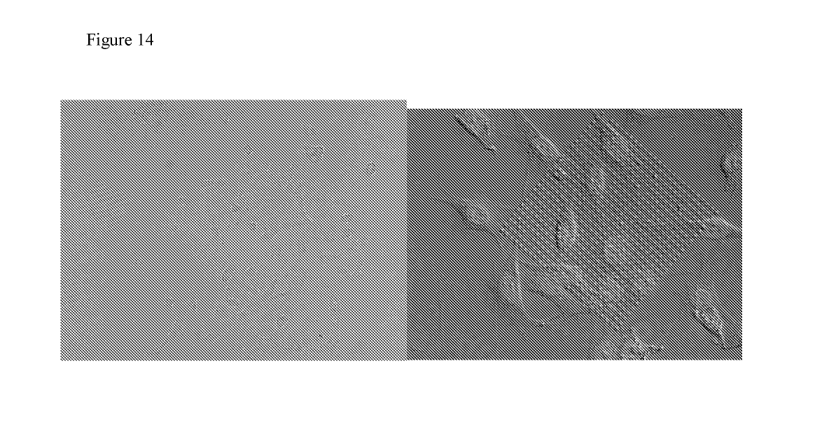

The next experiment demonstrates cell adhesion on PVA hydrogels; cf. FIG. 14. FIG. 14 simply to show that PVA hydrogels can be made cell adhesive. Pristine hydrogels do not support cell adhesion and cells “stop” in their proliferation at the border of the gel. In contrast, RGD-functionalized gels are well suited as substrates for cell adhesion. Importantly, this shows that SMEPT can be employed directly to the adhered cells and/or alternatively to achieve a drug delivery to the adjacent tissues.

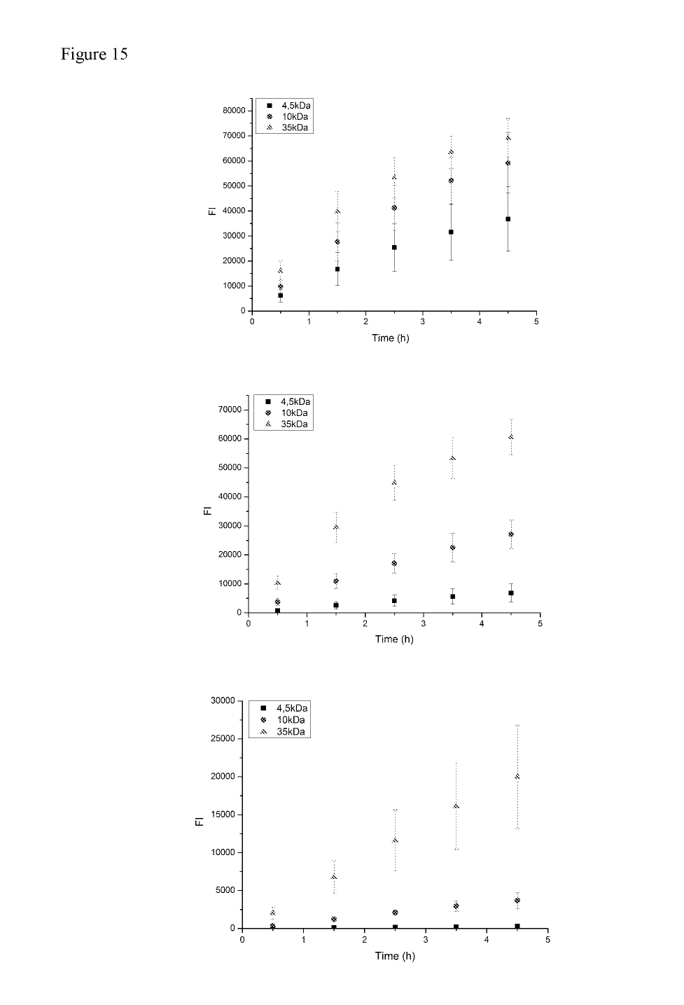

In the next experiment micro-structures of 4.5, 10 and 35 kDa of 12 wt % were loaded with enzyme, salted out for 1 h with 200 μL of 0.5M Na₂SO₄ and immersed into 800 μL PBS for different time periods of 1 h, 24 h, 3 days and 1week at 37° C., to evaluate the duration of enzyme activity after being immersed into long-term PBS. Different samples were made for each time period and the samples were analyzed for activity with 0.25 μg/mL fluorescein di-glucuronide. FIG. 15 shows the increase in fluorescence intensity due to converted substrate over a time period of 4.5 hour, with measurements taken each hour, and both 4,5, 10 and 35 kDa were analyzed.

FIG. 15 displays the enzyme activity in μS PVA hydrogels prepared using polymers with differed molecular weight. Substrate conversion was initiated in samples incubated in PBS for 1 h (top), 24 h (middle) and 7 days (bottom). Importance of this, further to the basic system characterization, lies in that it is shown that the SMEPT matrices can be made with time-programmed duration, i.e. life-time. It can be seen from FIG. 15 that 4 KDa looses activity essentially within a 24 h period. Furthermore, a pronounced difference between 10 and 35 KDa is found, and for the latter two, even after a week of incubation in PBS, there's a considerable level of enzymatic activity.

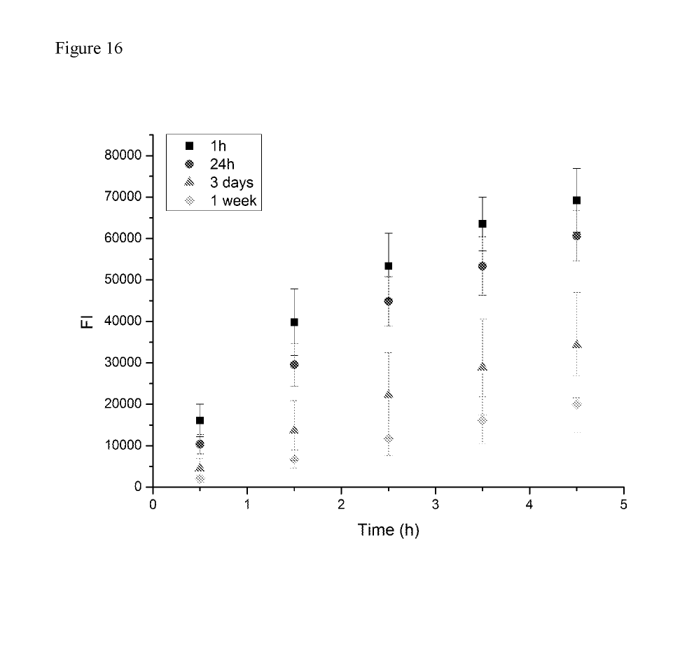

The next experiment illustrated in FIG. 16 address the enzyme activity for μS PVA hydrogels prepared using 35 KDa polymer sample with substrate conversion initiated at specified time points: 1 h, 24 h, 3 d and 7 d.

FIG. 16 shows that enzymatic conversion performed by the μS PVA hydrogels with immobilized b-Glu is preserved for at least a week. Thus, immobilized b-Glu is clearly suitable for use as an immobilized enzymes activity in a substrate of the present invention for use in SMEPT. Approximately 3-fold decrease in activity is acceptable in this system, as a decreased enzymatic activity can be offset by higher concentrations of the prodrug.

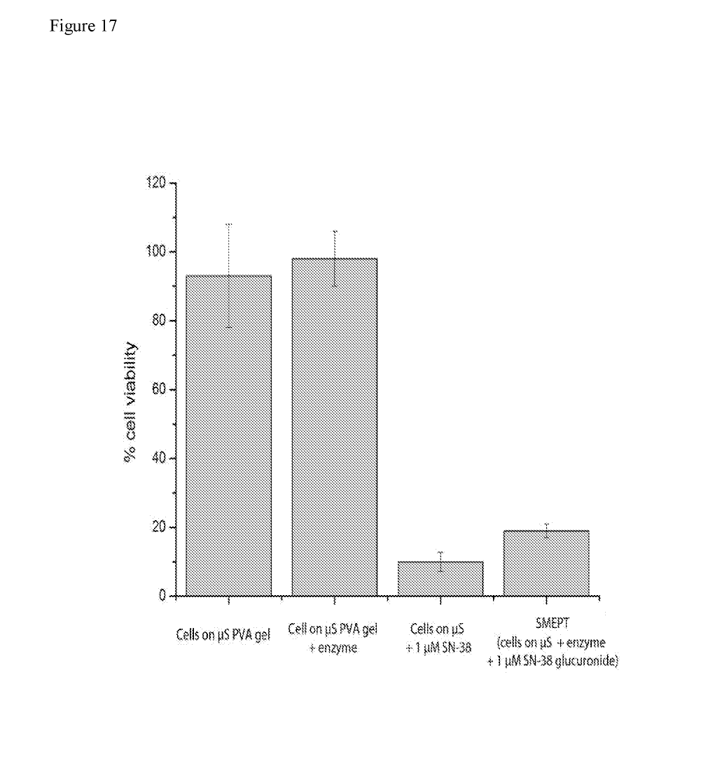

FIG. 17 shows the viability of cells on μS hydrogels with enzyme and/or prodrug. This experiment demonstrates the therapeutic applicability of SMEPT. The graphs shows: i) viability of the cells on the μS hydrogels; ii) same with immobilized enzyme; iii) viability of the cells cultured on μS PVA upon addition of 1 μM camptothecin SN-38, an anticancer drug; and iv) full SMEPT conditions, i.e. μS PVA with immobilized enzyme and 1 μM SN-38 glucuronide. It is seen that the glucuronidase enzyme immobilized in the hydrogel is capable of converting the provided SN-38 glucuronide prodrug into active agent with a cytotoxic effect which is comparable to the active drug SN-38.

### Example 4

Biocatalytic Polymer Coatings under Dynamic, Flow Conditions Cell Culture Afford On-Demand Drug Release and a Localized Therapeutic Effect

Introduction

The present example relates to the performance of enzyme-quipped Layer-by-Layer (LbL) thin films under flow conditions and uses this tool to illustrate unique opportunities associated with enzyme-prodrug therapy (EPT) approach in the context of surface mediated drug delivery (SMDD). Taken together, this example i) presents assembly of biocatalytic surface coatings based on multilayered polymer thin films, ii) demonstrates that EPT strategy engineered into surface coatings affords physiologically relevant concentrations of drugs, and does so under flow conditions, iii) illustrates that biocatalytic surfaces provide opportunity for on demand, “on-off” synthesis of the drug, and iv) reveals that this mode of drug delivery is indeed site specific.

The example demonstrates biocatalytic surface coatings for localized synthesis of the drug on the surface of implantable biomaterials and on demand, site-specific delivery of the therapeutic to adhering cells. This approach is based on incorporation of an enzyme into multilayered polymer coatings and performing enzyme-prodrug therapy (EPT) mediated by these versatile thin films. Build-up of enzyme-containing multilayered coatings is characterized, and the correlation is analysed between enzymatic activity of the resulting films and properties of the coating such as total mass of deposited polymers, dissipation value of the thin film, and total weight of incorporated enzyme. Therapeutic effect elicited by the surface mediated EPT (SMEPT) strategy is demonstrated using prodrugs for the anticancer agent, SN-38, and cells cultured on top of the biocatalytic surfaces. We illustrate performance of biocatalytic coatings under dynamic, flow conditions thereby demonstrating the utility of SMEPT as part of implantable biomaterials and “blood side mass transport” for applications such as re-endothelialization of grafts and stents. Specifically, it is demonstrated that EPT allows synthesizing the drugs on demand, at the time desired and in an amount to suit particular applications. Opportunity to control the concentration of the drug synthesized under flow conditions in unit time was investigated using such tools as concentration of the administered prodrug, content of the enzyme within the biocatalytic coating, and applied shear stress (flow speed). Finally, using cell culture in sequentially connected flow chambers, it is demonstrated that SMEPT affords a site-specific drug delivery system: SMEPT exerts a higher therapeutic effect in cells adhering directly to the biocatalytic coatings or in close proximity thereto than in the cells cultured “downstream”. Taken together, the data presented in this example illustrate unique biomedical opportunities made possible by engineering tools of EPT into multilayered polymer coatings and present a highly versatile, generic tool for surface mediated drug delivery.

Material and Methods

Materials Poly-L-Lysine (PLL, 30000-70000 Da), poly-L-Glutamic acid (PGA, 15000-50000 Da), poly(methacrylic acid) (PMA, 18600 Da), sodium alginate (ALG, Viscosity 5.0-40.0 cps, 1% at 25 C), 4-(2-hydroxyethyl)piperazine-1-ethane-sulfonic acid (HEPES), sodium chloride, and sodium dodecyl sulphate (SDS) were all purchased from Sigma Aldrich. Fluorescein di-glucuronide and PrestoBlue were obtained from Invitrogen. μ-Slides VI^{0,4} were purchased from Ibidi GmbH.

Unless stated otherwise, HEPES buffer was made to a concentration of 10 mM and 150 mM NaCl with pH 7.4. All buffer solutions were prepared using ultrapure water with a resistivity of 18.2 MΩ/cm obtained from a Milli Q Direct 8 system (Millipore). Poly(methacrylic acid)-co-(cholesteryl methacrylate) (PMA_{c}, Mn 33 kDa, D 1.05, 8 mole % cholesteryl methacrylate) was synthesized as described previously.⁵⁶

Methods

Polymer Synthesis and Dissolution:

Polymer solutions of PLL, PMA, PGA, and ALG were made at 1 g/L in HEPES buffer. PMAc was dissolved in DMSO (10 g/L) and diluted further to 0.25 g/L using HEPES buffer solution.

Quartz Crystal Microbalance with Dissipation monitoring (QCM-D). The assembly of polymer multilayers together with the enzyme were evaluated using QCM-D measurements (q-sense E4, Sweden). Silica-coated crystals (QSX303, q-sense) were immersed in a 2 wt % SDS solution overnight and subsequently rinsed with ultrapure water prior to blow-drying with N₂. The crystals were treated with UV/ozone for 20 minutes before placement in the liquid exchange chambers of the instrument. Both frequency and dissipation changes were monitored at a temperature of 24° C. After reaching a stable baseline in HEPES buffer solution, PLL (1 g/L) was introduced and left to adsorb onto the crystal until the surface was saturated. Buffer solution was introduced to rinse the chamber and remove excess polymer. A negative charged polymer (PGA 1 g/L, PMA 1 g/L, ALG 1 g/L, and PMAc 0.25 g/L) was then added to the chambers and after surface saturation HEPES buffer was used for rinsing. Alternating layers of PLL and the negatively charged polymer were deposited until 2.5 bilayers of the polymers were assembled. Then, the enzyme b-glucuronidase (20 μg/mL) was deposited followed by 2 bilayers of polymer starting with PMA. To optimize the enzyme adsorption conditions, the pH and salt content of the HEPES buffer solution containing the enzyme as well as the washing buffer before and after the enzyme deposition were varied.

Glass cover-slips. LbL assembly of PMA and PLL with incorporated enzyme was performed on glass cover-slips to evaluate the therapeutic effect of SN-38 under static conditions. Glass cover slips of 9 mm were sonicated in ethanol for 10 minutes, rinsed with ultrapure water, and dried with argon prior to UV sterilization for 20 minutes in a 48 well plate. Solutions of PLL and PMA, were incubated at 37° C. for 10 min prior to film assembly. Alternating layers were composed as (PLL/PMA)_{2.5}-b-glucuronidase-(PMA/PLL)₂ or (PLUPMA)_{4.5} for films without enzyme. Each layer was allowed to adsorb for 10 min at 37° C. followed by a washing step in HEPES buffer. The enzyme b-glucuronidase was added at a concentration of 20 μg/mL in HEPES buffer solution.

Perfusion Chambers. For flow conditions the I bidi μ-Slides VI^{0,4} coated with PLL were used to assemble the (PLL/PMA)_{2.5}-b-glucuronidase-(PMA/PLL)₂ film. The alternating layers were composed according to the protocol described for glass cover-slips above, without the first layer of PLL.

For evaluation of the enzymatic activity the prodrug fluorescein di-glucuronide, FdG, of 5 μg/mL was added to syringes and connected to the coated perfusion chambers. A syringe pump was used to control the shear stress using 0.15, 0.074, 0.03 dyn/cm². An “on demand” conversion of prodrug under flow conditions a syringe with buffer was used as a control. The “on” and “off” was then controlled by the switch of syringes to the given μ-slide chamber.

The influence of shear stress was evaluated by reducing the flow rate during the evaluation resulting in shear stresses decreasing from 0.15 to 0.074 and further to 0.03 dyn/cm².

For multiple layers of enzyme the PEMs were constructed as (PMA/PLL)₄ keeping the total amount of layers constant and adding the enzyme in between each layer of PLL.

All evaluations were measured out on the PlateReader by collecting aliquots from the outlet tubing during the experiment and transferred to a 96-well pate.

Cell Culture. The hepatocellular carcinoma cell line (HepG2) was used for all cell experiments. HEPG2 were cultured in a 75 cm² culture flask (3E6 cells/flask) in 10 mL medium (Minimum Essential Medium Eagle (MEME) supplemented with 10% fetal bovine serum (FCB), 1% penicillin and streptomycin, 1% non-essential amino acids, and 2 mM L-glutamine) and incubated at 37° C. and 5% CO₂.

Static cell culture. The LbL coated glass slides were UV sterilized for 10 minutes prior to cell seeding. HepG2 cells with a starting density of 75.000 cells in 300 μL were seeded per well. The cells were allowed to adhere overnight before adding the (pro)drug SN-38 of 1 μM and incubate for 48 h. 200 μL of fresh media was added together with 20 μL of PrestoBlue cell viability reagent and evaluated on an EnspireMultilabel Plate Reader after 30 minutes of incubation.

Dynamic cell culture in perfusion chambers. To perform experiments in the presence of shear stress, hepatocytes were seeded at a density of 30 000 cells/channel into closed perfusion chambers containing PEMs and allowed to attach overnight 37° C. and 5% CO₂. Subsequently, X mL of cell media containing 1 μM of (pro)drug SN-38 was added to the syringes (reservoir) as shown in Scheme X. The perfusion chambers were connected to syringe pump and placed in the incubator (37° C. and 5% CO2. The flow experiments were performed at a shear stress of 0.02 dyn/cm² for 4 hours. Thereafter, the channels were flushed with fresh media (2×) prior to futher incubation for 48 hours. and allowed to run for 4 h following 2× wash of the chambers with fresh media prior to 48 h incubation. The samples were evaluated by PrestoBlue on the Plate Reader after 30 minutes of incubation.

For the evaluation of drug delivery to subsequent chambers tubings were connected so that the flow of solution would enter the first chamber containing PEMs with enzyme and subsequent flow to chamber 2 and chamber 3 with cell culture on TCPS. Again, the μ-slide was kept at 37° C. in an incubator and the shear stress was set to 0,02 dyn/cm² (0.5 mL/h) and allowed to run for 4 h following 2× wash of the chambers with fresh media prior to 48 h incubation. The samples were evaluated by PrestoBlue on the Plate Reader after 30 minutes of incubation.

Results

Assembly of Biocatalytic Films

Assembly of polymer multilayers was quantitatively monitored using quartz crystal microbalance with dissipation monitoring. In the first experiment, we used one of the most well-studied pair of polyelectrolytes, synthetic polypeptides PLL and PGA. This pair has been studied towards fundamental understanding of LbL deposition of polymers and also used in biomedical applications for e.g. controlled cell adhesion and proliferation as well as drug delivery. As is typical for “weak” polyelectrolytes, deposition of these polymers can be controlled via pH of solution as well as the presence of low molecular weight electrolytes such as sodium chloride. These aspects of materials engineering are well documented in the state of art and herein, we used widely accepted deposition conditions, 1 g/L concentration of each of the polymers prepared using 10 mM HEPES buffer with pH 7.4 and supplemented with NaCl to 0.15 M. Deposition of each polymer layer was reflected by a decrease in resonance frequency of oscillation of the QCM crystals (Δf), cf. FIG. 18B. Following deposition of 5 polymer layers, the thin films were exposed to a solution of β-Glu (20 μg/mL) in aqueous buffers with pH values from 5 to 8.5. This pH range was monitored herein in an attempt to optimize adsorption of the protein through a variation of its ionization (for β-Glu, pl value is 4.8). Surprisingly, exposure of the polymer coated crystals to β-Glu afforded a rather small Δf (<10 Hz). This could be indicative of modest propensity of this protein for (electrostatic) adsorption onto polypeptide multilayered thin films terminated with PLL. Following the enzyme adsorption step, buffers within QCM chambers were changed back to HEPES, pH 7.4 and the adsorption of the polyelectrolytes was continued for another 4 layers as described above. For all protein deposition condition, the subsequent adsorption of the polymers remained well pronounced revealing that the enzyme immobilization did not hinder adsorption of additional polymer layers. Adsorption of capping polymer layers also exhibited no obvious trend with regard to the adsorption conditions of β-Glu: polymer layers were characterized with the lowest value of Δf following protein adsorption at pH 7.4, revealed the highest Δf when the enzyme was introduced at pH 5, and intermediate for pH of protein solutions 6 and 8.5.

The top three panels in FIG. 19 show a summary of the (PLL/PGA)_{2.5}/β-Glu/(PGA/PLL)₂ film assembly as assessed by QCM-D. The first and second panels from the top represent the overall ΔD and Δf for the entire film assembly, showing similar values for ΔD for all the tested pH conditions during the enzyme adsorption. On the other hand, the total Δf for pH 6 and 7.4 were similar and lower than for pH 5 and 8.5, indicating that more hydrated films were deposited in the latter case. The third panel from the top displays Δf for the enzyme deposition step, showing minor changes in all cases, as discussed above. In order to further characterize the multilayered films, we measured the enzymatic activity of the biocatalytic film and compared the findings to the QCM-D results. To assess enzymatic activity, the crystals with the adsorbed polymer films were exposed to a 0.5 pg/mL solution of a fluorogenic substrate, fluorescein diglucuronide (FdG), conversion of which affords a highly fluorescent product, fluorescein. Incubation was conducted within the QCM chambers for 30 min, following which reaction volumes were collected, and fluorescent intensities thereof were used to quantify the concentration of produced fluorescein using a standard curve. Multilayered (PLL/PGA)_{4.5} films were used as an enzyme-free control. FIG. 19D reveals that the deposition of the enzyme at pH 7.4 yielded the highest enzymatic conversion followed by pH 8.5 during enzyme adsorption. The other two tested pH values did not lead to increased fluorescent intensity of the solution as compared to the control, indicating either negligible amounts of adsorbed protein or enzyme deactivation. It is important to emphasize that following the protein deposition step, the chambers were repeatedly washed and exposed to polymer solutions for subsequent deposition steps. This implies that enzymatic conversion was achieved by the enzyme immobilized within the coatings (deposited on the crystal and the chamber interior) whereas non-adsorbed protein was removed from the chamber during numerous washing steps. The main observation from the data presented in FIG. 19 is that with the appropriate deposition conditions, multilayered thin films act as appropriate matrix for catalytically active β-Glu and reveal pronounced enzymatic activity. This conclusion is encouraging for the development of SMEPT based on LbL coatings, and is quite surprising given the small Δf measured during the enzyme deposition step (FIG. 19C). Further conclusion is that the measured Δf for the enzyme adsorption and the resulting enzymatic activity reveal no correlation, and conditions optimal for the highest resulting biocatalytic activity are not those which afford the most pronounced protein deposition. Similarly, comparing the enzymatic activity of the films to the overall ΔD and Δf of the assembled coatings, also exhibited no correlation. These data is important for the development of enzyme-containing multilayered polymer coatings for EPT and other biocatalytic applications.

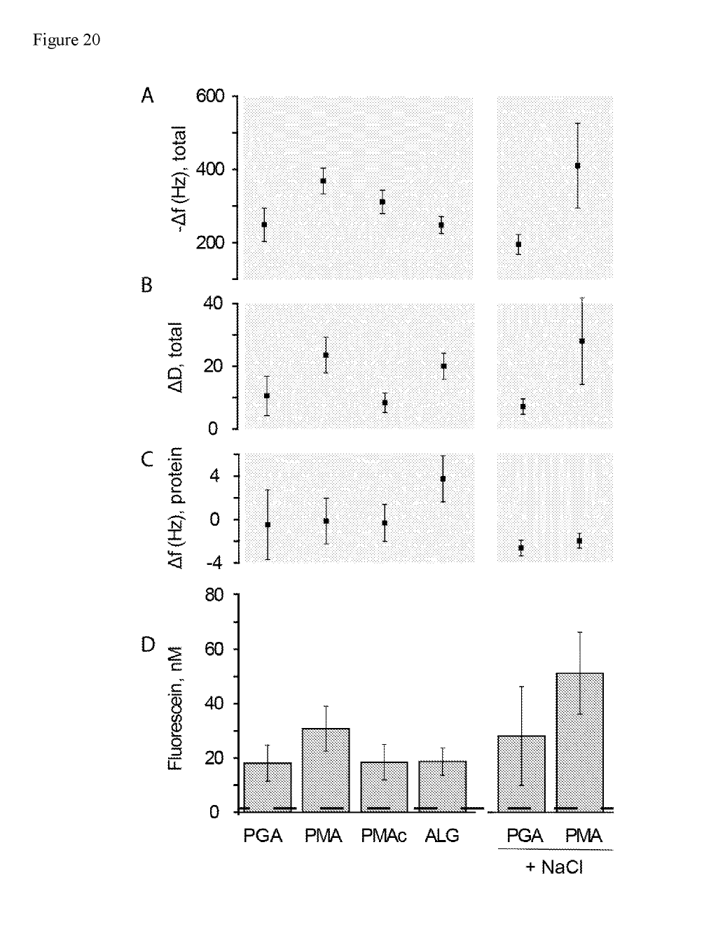

To further investigate the assembly of the biocatalytic multilayered thin films, polymer coatings were assembled using PLL as a cationic polyelectrolyte and replacing the anionic polymer PGA with three different counterparts (P-), ALG, PMA, and cholesterol-modified PMA (PMAc). The polymer and enzyme deposition was monitored via QCM-D, and the enzymatic activity of the films was ascertained as described above using FdG. The protein adsorption was accomplished from HEPES buffer pH 7.4 as these adsorption conditions revealed the highest level of enzymatic activity (FIG. 18). Data on the total ΔD and Δf of the deposited films, Δf adsorbed protein, and levels of enzyme-mediated substrate conversion are shown in FIG. 20 depending on the used anionic polymer in the (PLL/P-)_{2.5}/β-Glu/(P-/PLL)₂ assembly.

The main conclusion from this data set is that for each combination of the polymers, resulting coatings afforded enzymatic conversion of the substrate, and therefore could serve as matrices for EPT applications. This holds true for all-natural multilayered films and those made using synthetic polyanions, as well as cholesterol-functionalized polymer coatings.

Of these, PLL/PMA pair polymers produced the films with the highest total Δf and ΔD, and all the other tested combination were similar and lower. Finally, Δf of the polymer pre-coated crystal due to the exposure to the protein solution was most pronounced PLL/ALG coatings. However, none of the above polymer thin film characteristics (total/protein Δf or ΔD) revealed a correlation with the enzymatic activity of the assembled films. Data in FIG. 20 shows that enzymatic activity is determined by local environment of the protein and may not be readily predicted based on the properties of the polymer film. This conclusion finds further support in the results of optimization of protein activity towards which for selected polymer pairs, PLL multilayers with PGA and PMA, enzyme immobilization was achieved using HEPES buffer in the presence of 0.15 M NaCl. PGA was employed since it is a degradable polypeptide and PMA containing films showed the highest enzymatic activity in the absence of NaCl. The monitored total/protein Δf or ΔD with or without 150 mM NaCl during enzyme deposition were similar. Further, while (PLL/PGA)_{2.5}/β-Glu/(PGA/PLL)₂ films exhibited similar biocatalytic activity independent on the ionic strength during the enzyme adsorption, the biocatalytic activity of (PLL/PMA)_{2.5}/β-Glu/(PGA/PMA)₂ coatings was ˜2× higher when the enzyme was deposited in the presence of 150 mM NaCl.

SMEPT Using Static Cell Culture

(PLL/PMA)_{2.5}/β-Glu/(PGA/PMA)₂ films revealed the highest level of enzymatic activity, and these polymer coatings were used to investigate the therapeutic effect of these biocatalytic thin films. A glucuronide prodrug of a commercial anticancer drug, SN-38, was used. For cell culture experiments a hepatocyte cell line, HepG2, was used, which also serves to illustrate the potential of SMEPT in hepatic drug delivery. Multilayered polymer coatings have been previously investigated in the context of hepatic applications (tissue engineering) and would benefit from advanced tools of drug delivery such as EPT. Further, hepatocellular carcinoma (HCC) is a condition successfully addressed using tools for localized drug delivery, but solitary drugs are currently accommodated for this mode of delivery. Multilayered coatings and tools of EPT provides versatility in drug delivery and/or imaging of HCC.

Hepatocytes were seeded on the biocatalytic film (PLL/PMA)_{2.5}/β-Glu/(PGA/PMA)₂ and allowed to adhere overnight before refreshing the media and adding the (pro)drug at 1 μM. After 48 h incubation, cell viability was assessed and found to be reduced by ˜80% when compared to cells adhering to the control (tissue culture polystyrene well plates) (FIG. 21A) or cells seeded on the polymer films without the presence of the (pro)drug (FIG. 21E). This reduction in viability of the hepatocytes was found to be similar as for hepatocytes adhering to (PLL/PMA)x films upon the administration of the prodrug and the enzyme (FIG. 21B) or the parent drug SN-38 (FIG. 21C), i.e. solution based EPT or traditional drug administration. Administering the prodrug to hepatocytes adhering to (PLL/PMA)_{4.5} coatings (no enzyme incorporated, FIG. 21D) resulted in a minor albeit statistically significant decrease in the cell viability. This observation was surprising in that we have previously shown that SN-38 glucuronide did not have an inherent cytotoxic effects on hepatocytes. Taken together, data in FIG. 21 illustrate utility of SMEPT for therapeutic intervention mediated by a biocatalytic surface coating and accomplished through an external administration of a prodrug.

Enzymatic Conversion in Biocatalytic Films in the Presence of Shear Stress.

In order to demonstrate the abilities of EPT to synthesize and locally deliver the drugs ‘on demand’ and specifically to realize “on-off” dosage, a microfluidic set up was employ. Testing the biocatalytic films under dynamic conditions, i.e. within flow chambers with biomaterials tested under constant flow of media, provides a markedly better mimic of the in vivo settings wherein the coatings are surrounded by flowing blood, lymph, interstitial fluid, etc. For SMDD systems, constant fluid exchange may arrest the success of therapeutic intervention through eliminating the released drug from the site of release and into systemic circulation. However, to the best of our knowledge, there are solitary attempts on verifying drug release mediated by implantable biomaterials under flow conditions. For EPT strategies, unlike conventional SMDD approaches, flow conditions are highly beneficial in ensuring a constant supply of prodrugs and thus, contributing to biocatalytic synthesis of the drug. Furthermore, supply of the prodrug is a strict requirement for EPT which implies that in absence of the prodrug, EPT coating does not generate the product forming a foundation for an ‘on demand’ synthesis of the drug.

To demonstrate this, the biocatalytic (PLL/PMA)_{2.5}/b-glu/(PGA/PMA)₂ films were assembled within commercially available PLL pre-coated perfusion chambers designed for cell culture under flow conditions. Polymer and protein deposition was accomplished using PLL and PMA as constituting polymers and b-Glu as the enzyme at conditions as described above. Assembly was performed using identical chambers which were connected to supply of prodrug containing solution of “placebo”, prodrug-free phosphate buffer saline, cf. FIG. 22. Administration of a model prodrug (fluorescein diglucuronide) into chamber A resulted in the synthesis of the product, fluorescein, which was readily detected by fluorescence measurements performed on collected volumes at the chamber outlet. In contrast, “placebo” (PBS) solution administered into chamber B resulted in negligible solution fluorescence indicative of the absence of synthesis of the fluorescent product. At arbitrary time points, administration solutions were swapped to feed the prodrug into chamber B and PBS solution into chamber A. Upon switching, volumes collected at the outlet of chamber A revealed minimal fluorescence, while those collected from chamber B demonstrated pronounced fluorescence indicating successful generation of the fluorescent product. Placebo and prodrug feeds were swapped several times and in each run, placebo feed resulted in a negligible solution fluorescence whereas feeding FdG afforded the expected synthesis of fluorescein. It is imperative that with constant concentration of the prodrug in the feed solution, amount of the product generated by the biocatalytic coatings is well reproducible and remains near-constant throughout the observation time, a feature of highest importance for reliable drug dosage. These results illustrate the desired ‘on demand’ synthesis of the product accomplished by a model implantable biomaterial, and further demonstrates a facile “on-off” drug dosage achieved using biocatalytic film designed for SMDD.

In order to demonstrate control over the enzymatic conversion of the prodrug depending on the testing conditions or the composition of the biocatalytic film, the applied shear stress (flow rate of the medium), the concentration of the administered prodrug, and the amount of entrapped enzyme within the films was varied. All the other parameters of the system but the nominated factor were kept constant and these data present the tools of control over enzymatic conversion of the prodrug and/or parameters that have to be taken into account when designing specific applications of the system.

To investigate the influence of shear stress on the performance of biocatalytic coatings, solution of the fluorogenic prodrug was administered into the flow chambers at varied flow rate, from 1 to 5 mL/h which corresponds to shear stresses from 0.03 to 0.15 dyn/cm². Accelerated feed of the prodrug solution over enzyme-containing coating resulted in a progressively lower amount of the product synthesized in unit time, possibly through a decreased residence time of the prodrugs and the enzyme. This observation as such is rather expected. Therefore, it should be noted that in order for an EPT strategy to accommodate e.g. cardiovascular applications, EPT/LbL coatings should be optimized to be performed at shear stresses associated with the blood flow within a human body. However, such optimization is achievable by regulating other features of the strategy, such as the concentration of immobilized enzyme in the coating and/or the amount of prodrug provided. However, in the form described in the present example, the EPT strategy is useful when engineered into stents for other, non-cardiovascular applications as well as other implantable biomaterials, such as embolic bodies. Thus, shear stresses as applied in this experiment are similar to those encountered in the liver and data presented herein illustrate that at these flow rates, designed coatings are effective in in-the-flow conversion of the prodrugs into their products.

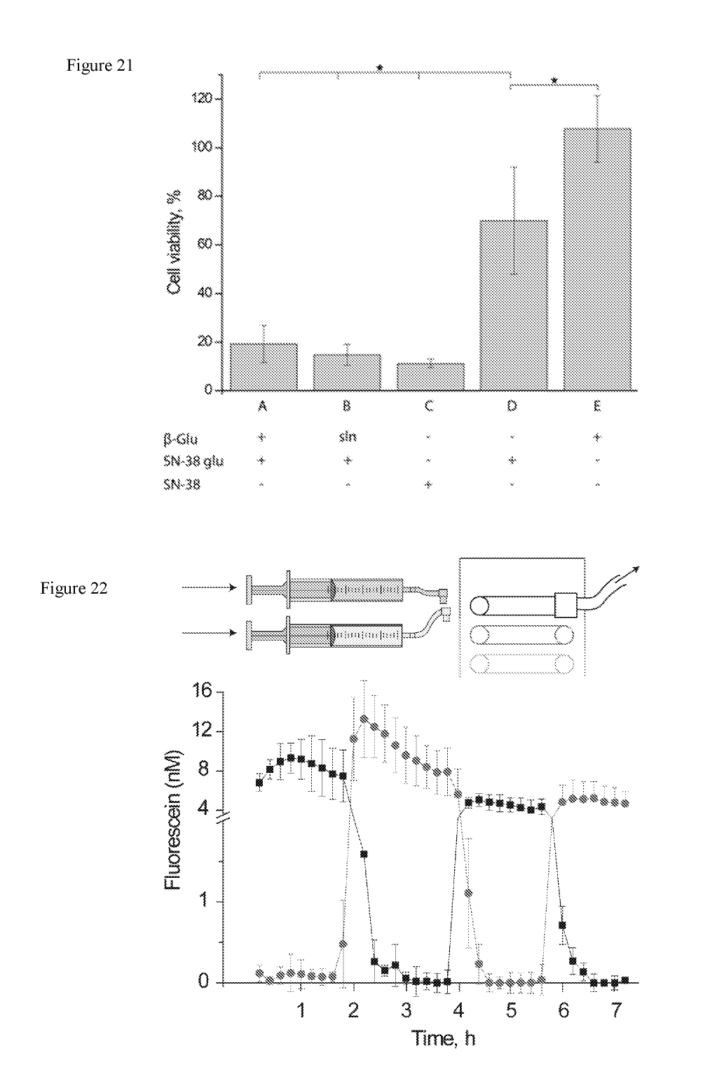

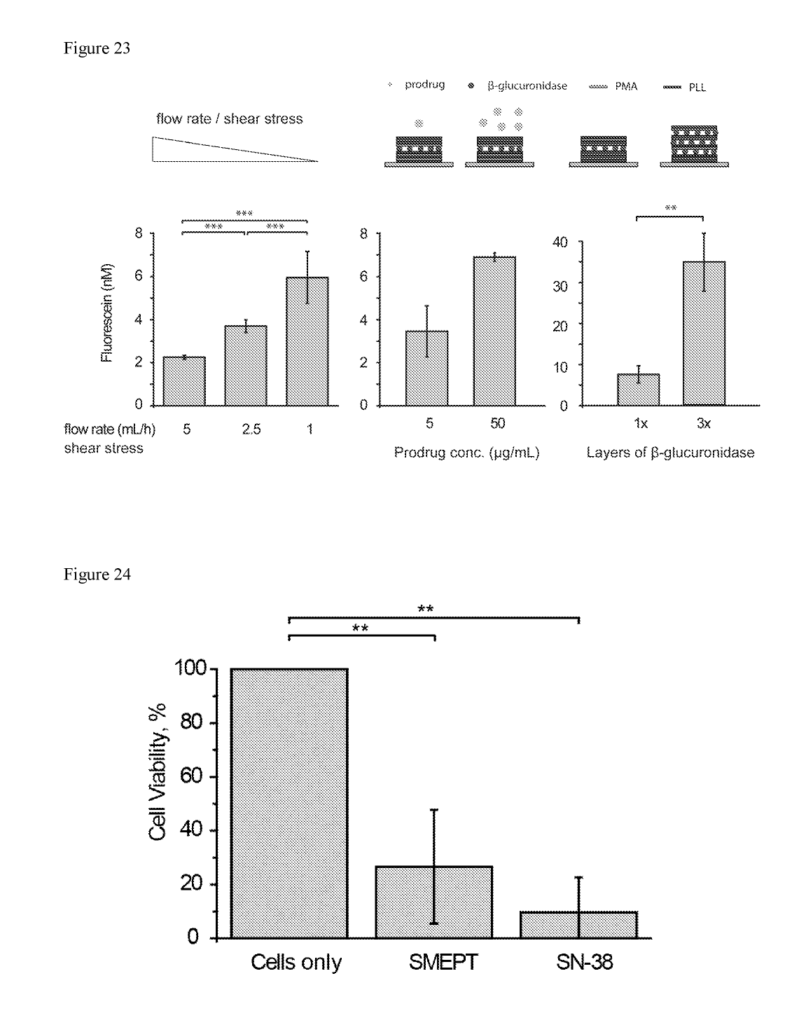

When exposing the biocatalytic (PLL/PMA)_{2.5}/b-glu/(PGA/PMA)₂ films to a higher concentration of the prodrug, the monitored amount of the converted product in the presence of shear stress increased as expected (FIG. 23B). This shows control over the rate of enzymatic conversion and the synthesis of the reporter product and illustrates control over SMEPT using tools inherent with enzymatic biocatalysis. A unique opportunity associated with multilayered surface coatings relates to the possibility to build films with a desired thickness for which polymer adsorption can be performed to a desired number of layers. Similarly, LbL deposition would allow controlling the amount of enzyme within the thin film through a facile change in the number of protein deposition steps. To show this, polymer coatings were assembled using one or three layers of adsorbed β-Glu keeping the total number of polymer layers constant. Administration of solutions with identical concentration of the prodrug to the chambers with 1 or 3 layers of adsorbed protein resulted in a pronounced, statistically significant, approximately 3× higher amount of product synthesized in unit time. While a single layer of the enzyme achieved a <10 nm concentration of the product, three protein layers suffice for a build-up of product concentration to over 30 nM, all achieved under dynamic, constant flow conditions. This result demonstrates the possibility to tune the biocatalytic activity of the multilayered surface coating through assembly of architectures with appropriate, rationally designed content of the enzyme.

To demonstrate the therapeutic potential of SMEPT under dynamic conditions, perfusion chambers were coated with the biocatalytic (PLL/PMA)_{2.5}/b-glu/(PGA/PMA)₂ films and used as substrates to culture hepatocytes. In preliminary experiments, it was verified that a well-pronounced therapeutic effect of SN-38 requires at least 48 h of cell culture. However, the presence of the drug (1 μM SN-38) was required for as little as 4 h, and subsequent culture in drug-free media also resulted in a pronounced therapeutic effect. Based on these findings, cell culture in surface-modified flow chambers was conducted in the presence of the (pro)drug (1 μM) and under flow conditions over the initial 4 h after which subsequent cell culture was performed at static conditions in drug free medium. Following a total of 48 h of culture, chambers were filled with a reagent to measure cell viability (PrestoBlue, Invitrogen) and collected volumes were analysed on a multi-label plate reader. Cell viabilities were normalized to cells cultured at in the absence of (pro)drug in the media. Administration of 1 μM glucuronide of SN-38 resulted in a statistically significant decrease in cell viability by ˜70%, thus verifying the therapeutic effectiveness of the assembled biocatalytic surface coatings. We note that while no attempts was made to culture the cells to confluency, results in FIG. 24 reveal that diffusion of the prodrug into the coating and release of the resulting product are not limited by the cells adhering to the surface of a biomaterial, and within cell culture experiments, biocatalytic thin films afford a pronounced therapeutic effect.

To further investigate opportunities made available by biocatalytic surface coatings, the potential of SMEPT was examined for site-specific drug delivery as an attribute of therapeutic implantable biomaterials and SMDD systems. With rare exceptions, characterization of LbL coatings and other tools of surface mediated drug delivery were typically conducted under static release and cell culture conditions. Successful examples of localized drug delivery from model surfaces achieved under static conditions may relate to the so called “wall side mass transport”, i.e. scenario mimicking delivery of the drug from the surface of a cardiovascular stent into the blood vessel wall or from an implant into surrounding tissue. In contrast, static test conditions are hardly appropriate to mimic “blood side mass transport” with specific relevance to drug release into the blood stream. The latter scenario is highly relevant in the context of re-endothelialization of grafts and stents wherein released drugs aim to mediate adhesion of platelets, proliferation of myoblasts and endothelial cells etc.—on the blood side of the stent or graft. Nevertheless, opportunities in investigating the “blood side mass transport” and specifically cellular responses under flow conditions are developed significantly less than approaches in static cell culture. Flow chambers as employed in this work and a possibility to deposit the multilayered coatings inside the chambers prior to cell seeding present themselves as a convenient platform to investigate cellular responses achieved through surface mediated drug release.

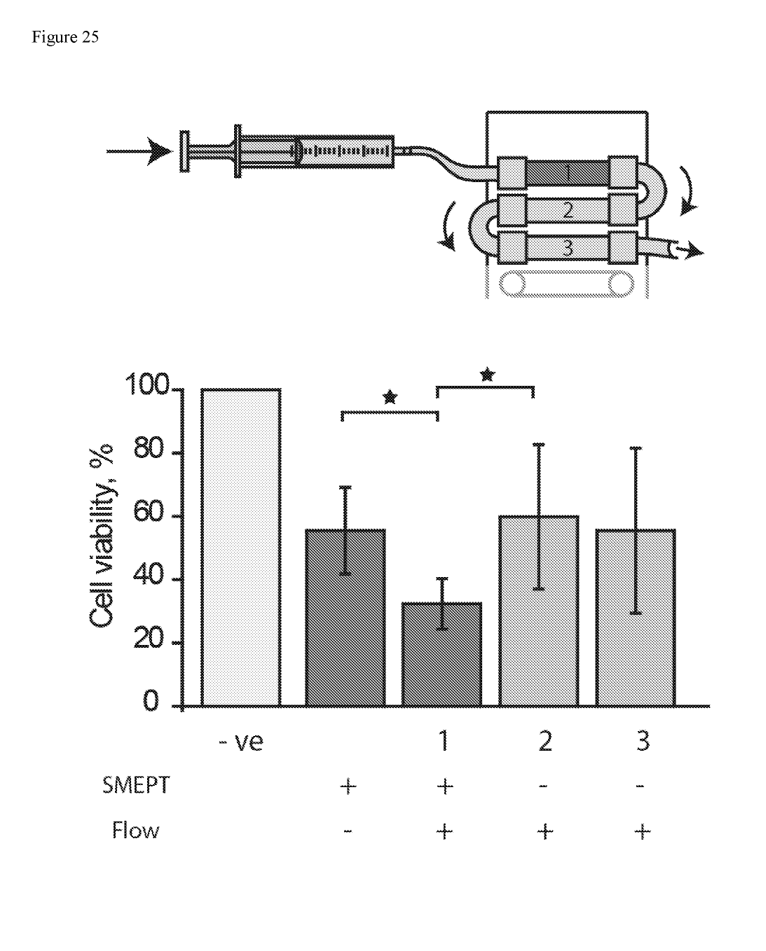

It is assumed that when the drug is synthesized within the coating immediately underneath the cultured cells, through increased local concentration of the drug, the synthesized therapeutic would exert a higher local effect in adhering cells as compared to the cells growing “downstream”. To test this, biocatalytic (PLL/PMA)_{2.5}/b-glu/(PGA/PMA)₂ multilayered biocatalytic coatings was deposited within the first of three sequentially connected perfusion chambers (FIG. 25A). Outlet from chamber 1 was connected to the inlet for chamber 2, and in turn, outlet of chamber 2 was connected to the inlet of chamber 3. Hepatocytes were seeded in all three chambers, but only the cells in chamber 1 were cultured on biocatalytic coatings. Solution of SN-38 glucuronide (1 μM in cell culture media) was administered to the interconnected perfusion chambers at constant shear stress for 4 h. Subsequently, the perfusion chambers were flushed with (pro)drug-free media and the cells were cultured for a further 48 h, prior to the assessment of the cell viability.

The viability of cells cultured on biocatalytic coatings was reduced to ˜30% verifying a pronounced therapeutic response achieved via SMEPT under flow conditions. Viability of cells in chamber two was reduced to ˜60% thus being much higher than that for cells cultured on biocatalytic coatings. Taking into account dose response of HepG2 cells to SN-38, viability difference between 30% and 60% corresponds to ˜10-fold difference in the concentration of the drug used for the treatment. This notion illustrates a significantly higher local concentration of the drug created locally through an EPT approach. Note that the flow chambers setup is an important step towards in vivo conditions but does not take into account a rapid dilution of the drug in the blood flow. It is therefore highly likely that local therapeutic effect would exceed that at “downstream” location to a greater extent than illustrated in FIG. 25. Our data provide experimental proof of the site-specific drug delivery achieved via SMEPT and thus, illustrate therapeutic advantage associated with this technique.

### Example 5

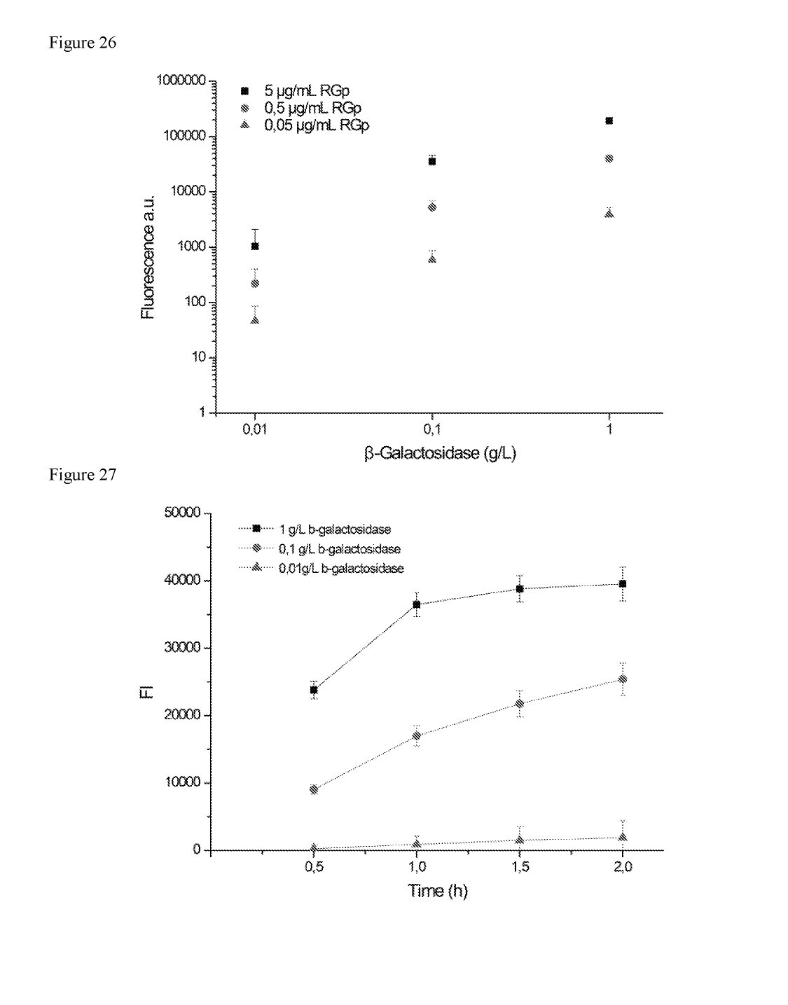

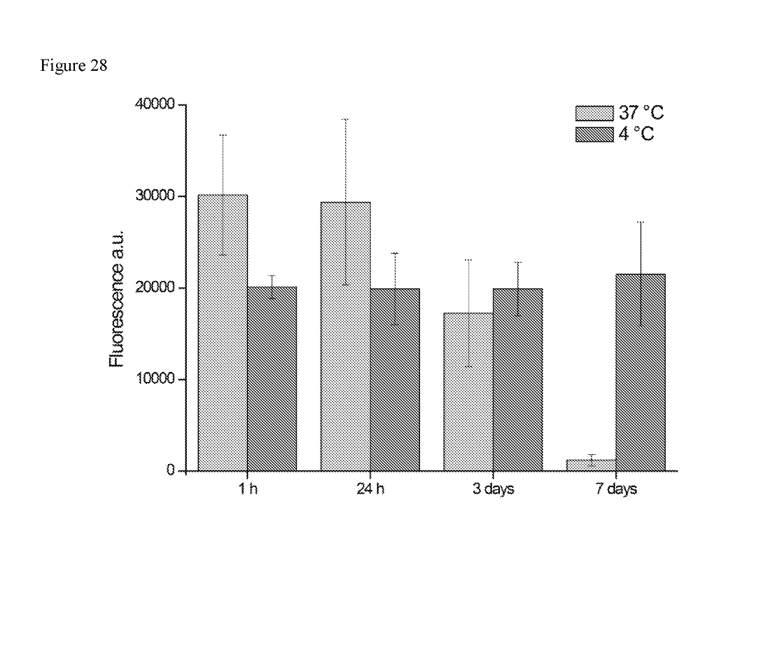

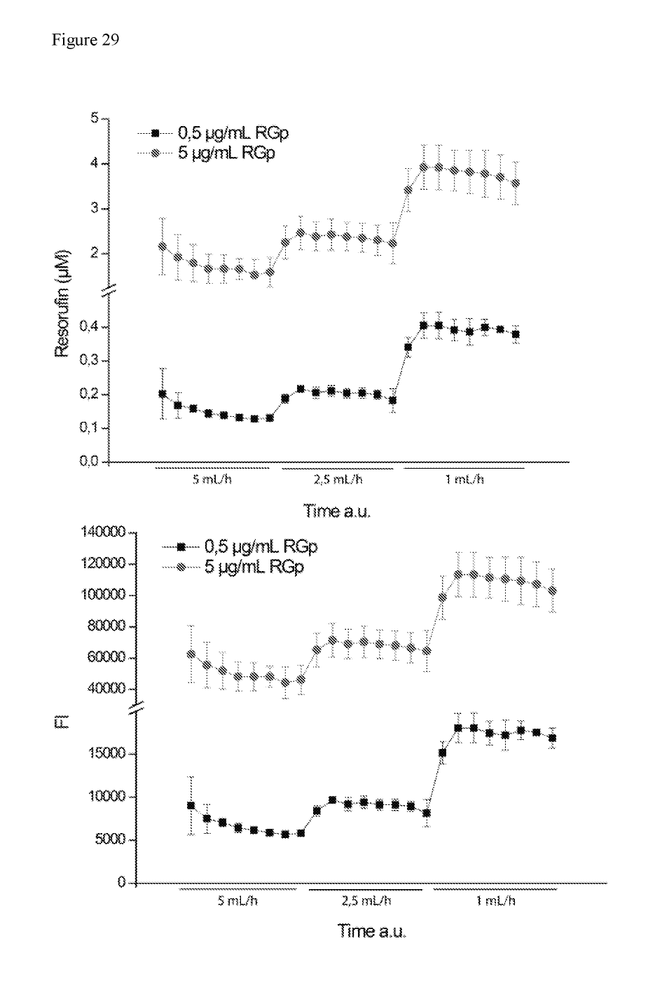

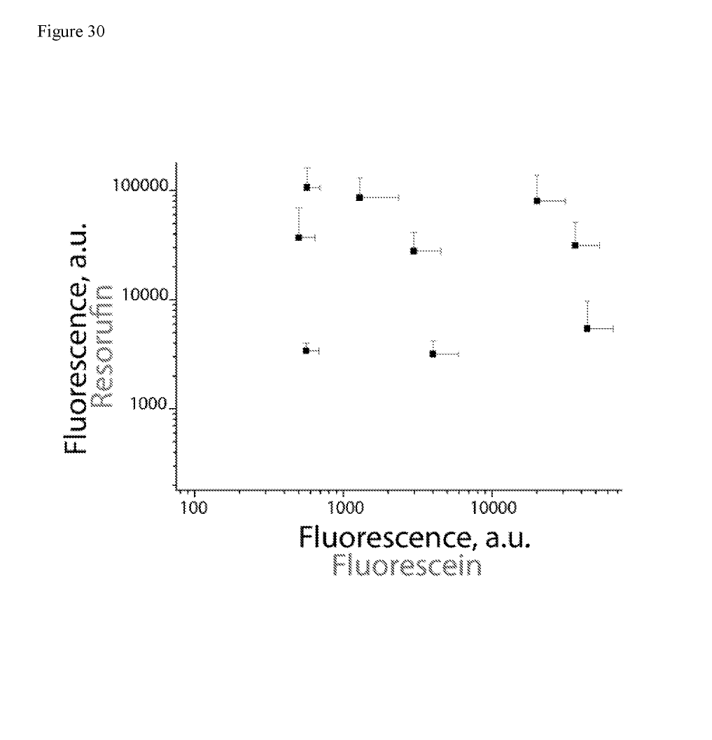

FIGS. 26-30 demonstrate different features of SMEPT using β-Galactosidase in PVA hydrogels, including the flexibility of the SMEPT technology in terms of varying the concentration of the enzyme within the hydrogel and concentration of the administered prodrug provide two independent tools to control the amount of the active drug product generated in unit time (FIG. 26); constant rate of prodrug conversion/drug production at specific enzyme and prodrug concentrations (FIG. 27); shelf life of SMEPT substrates (FIG. 28); the correlation between flow rate and enzyme concentration (FIG. 29); and multiplexing SMEPT by providing multiple prodrugs (FIG. 30).

### Example 6

Enzymatic Conversion of Different Concentrations of the Prodrug β-gal-NONOate to Nitric Oxide (NO) by the Enzyme g-Galactosidase

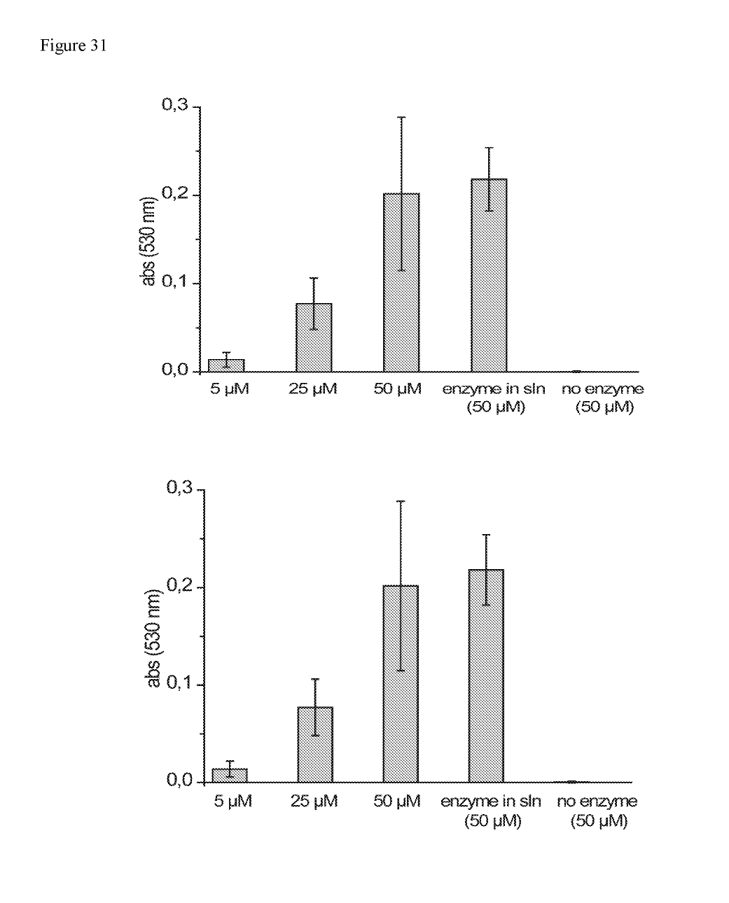

Microstructured PVA hydrogels were assembled using polymer solutions containing b-Galactosidase (1 g/L), stabilized using 0.5 M sodium sulfate and incubated in PBS for 1 h prior analysis. We used a substrate for b-Gal conversion of which affords nitric oxide (NO), a potent and multi-functional signaling and therapeutic molecule. This radical—molecule has a very short half-life and is rapidly converted into nitrite/nitrate, and the latter are conventionally used to quantify the levels of generated nitric oxide. Analysis of nitrite is conducted via a Griess assay, i.e. a colorimetric test establishing a linear correlation between the concentration of nitrite and absorbance at 530 nm; cf. FIG. 31.

b-Gal-NONOate was administered onto the enzyme-functionalized hydrogels at varied concentrations and incubated for 30 min, upon which the levels of nitrate were quantified via the Griess assay. Biocatalytic hydrogels is found to afford a rapid conversion of the prodrug into the product, nitric oxide, converting approximately 50% of the administered dose within 30 min of incubation.

This data illustrates the utility of SMEPT for generating NO for diverse applications, specifically cardiovascular stenting and re-endothelialization of stents and grafts. Note that also for generation NO by SMEPT, conversion depends on the prodrug concentration, and that there is a positive correlation between the concentration of prodrug and the amount of drug (NO) generated. The conversion of prodrug in SMEPT is similar to that observed in EPT. Moreover, SMEPT conversion is specific and reliable, as no prodrug conversion/drug production of NO is observed in the absence of enzyme.

1. A method of administering at least one active drug agent to cells adhering and/or adjacent to a substrate, said substrate comprising one or more immobilized enzymes, which are capable of converting an inert prodrug into an active drug agent, said method comprising providing at least one inactive prodrug to said substrate.

2. (canceled)

3. A method of treating a disease in an individual, said method comprising localizing a substrate comprising one or more immobilized enzymes capable of converting an inert prodrug into an active drug agent to a target site for treatment, and systemically administering an efficient amount of at least one said inert prodrug to said individual, thereby bringing said at least one prodrug into contact with said one or more enzymes and thereby converting said inert at least one prodrug into active drug at the target site for treatment.

4. The method according to claim 3, wherein said disease is atherosclerosis and said method comprises inserting a vascular stent, such as vascular graft, comprising a substrate comprising one or more immobilized enzymes capable of converting an inert prodrug into an active drug agent, into a blood vessel, and administering at least one inert prodrug, which is converted into active anti-coagulating drug agent.

5. The method according to claim 1, wherein said substrate is a hydrogel.

6. The method according to claim 1 wherein said hydrogel is a polyvinyl alcohol (PVA) hydrogel.

7. The method according to claim 1, wherein said substrate is selected from the group consisting of polymers, polymer thin films, and porous solid matrices.

8. The method according to claim 1, wherein said substrate is selected from Poly-N-isopropylacrylamide (pNIPAAM), Polyethylene glycol (PEG), and Alginate.

9. The method according to claim 1, wherein said substrate is a material having a surface comprising sequential polymer deposition (layer-by-layer coating).

10. The method according to claim 9, wherein said surface comprises sequential layers of polymer pairs selected from the group consisting of poly(L-lysine) (PLL)/Alginate, PLL/poly(L-glutamic acid) (PGA), thiol-modified poly(methacrylic acid) (PMAsh)/poly(N-vinylpyrrolidone) (PVP) and poly(allylamine hydrochloride) (PAA)/poly(acrylic acid) (PAH).

11. The method according to claim 1, wherein said substrate is part of or is a stent, a vascular graft, an implantable device, a scaffold for tissue engineering.

12. The method according to claim 1, wherein said one or more immobilized enzymes are selected from the group consisting of glucuronidase, glycosidase and beta-lactamase.

13. The method according to claim 1, wherein said enzyme is β-glucuronidase.

14. The method according to claim 1, wherein said prodrug is a glucuronide-conjugated prodrug.

15. The method according to claim 1, wherein said active drug agent is a growth hormone, a cytotoxic drug agent, an anti-inflammatory drug agent, an antiviral drug agent, an anti-platelet aggregation drug agent, such as clopidogrel or an imaging agent.

16. The method according to claim 1, wherein said enzyme is β-glucuronidase and said prodrug is a glucuronide prodrug.

17. The method according to claim 1, wherein 1, 2, 3, 4, or 5 different inert prodrugs are administered.

18. The method according to claim 1, wherein said substrate comprises 1, 2, 3, 4, or 5 different enzymes.

19. The method according to claim 1, wherein said inert prodrug is provided temporally only when the action of the active drug agent is required to be administered to said cells adhering and/or adjacent to said substrate.

20. The method according to claim 1, wherein the amount of said one or more provided inert prodrugs reflects the level of active drug agent to be administered to said cells adhering and/or adjacent to said substrate.

21. A kit comprising a substrate comprising one or more immobilized enzymes capable of converting an inert prodrug into an active drug agent, and at least one inert prodrug.

22. (canceled)

23. (canceled)

24. An implantable device comprising a substrate comprising one or more immobilized enzymes capable of converting an inert prodrug into an active drug agent.

25. The implantable device according to claim 24, wherein said device is a stent comprising a substrate comprising one or more immobilized enzymes capable of converting an inert prodrug into an active drug agent.

26. The implantable stent according to claim 25, wherein said stent is a cardiovascular stent, a bowel stent, a ureteral stent, a prostatic stent, an esophageal stent, a biliary stent, a stent graft or a vascular graft.

27. (canceled)

28. (canceled)
